(19) United States
(12) Patent Application Publication
(10) Pub. No.: US 2024/0238317 A1
(43) Pub. Date: Jul. 18, 2024

(54) METHODS FOR REDUCING INTRAOCULAR PRESSURE

(71) Applicant: UNIVERSITY OF MISSISSIPPI, University, MS (US)

(72) Inventors: Soumyajit Majumdar, Oxford, MS (US); Narendar Dudhipala, Woodbridge, NJ (US); Mahmoud A. Elsohly, University, MS (US); Waseem Gul, University, MS (US)

(21) Appl. No.: 18/559,887

(22) PCT Filed: May 12, 2022
(86) PCT No.: PCT/US22/72287

§ 371 Date: Nov. 9, 2023

Related U.S. Application Data
(60) Provisional application No. 63/187,472, filed on May 12, 2021.

(51) Int. Cl. A61K 31/00; A61K 9/00; A61K 9/107; A61K 31/472; A61K 47/10; A61K 47/22; A61K 47/44; A61P 27/06

(52) U.S. Cl. CPC A61K 31/658; A61K 9/0048; A61K 9/1075; A61K 31/472; A61K 47/10; A61K 47/22; A61K 47/44; A61P 27/06

(57) ABSTRACT

Described herein are methods for reducing or preventing intraocular pressure in a subject. The methods involve administering to the subject a delta-9-tetrahydrocannabinol amino acid ester or derivative thereof and the Rho kinase inhibitor. The combination of delta-9-tetrahydrocannabinol amino acid ester or derivative thereof and the Rho kinase inhibitor is effective in reducing intraocular pressure when compared to independently the delta-9-tetrahydrocannabinol amino acid ester or derivative thereof and the Rho kinase inhibitor.

### CROSS REFERENCE TO RELATED APPLICATIONS

This application claims the benefit of U.S. Provisional Application No. 63/187,472 filed on May 12, 2021, which is incorporated herein by reference in its entirety.

### BACKGROUND

Delta-9-tetrahydrocannabinol (THC) is the primary active ingredient of the plant Cannabis sativa (marijuana) and is responsible for the majority of the pharmacological effects. People have utilized the plant (that includes numerous cannabinoids) since ancient times for medicinal purposes as well as for its intoxicating properties. While marijuana is primarily known as an abused drug, there are important pharmacological properties of the active component THC that could be directed to specific therapeutic effects, given the appropriate delivery mechanism. To date, the most promising clinical applications approved by the Food and Drug Administration (FDA) are for the control of nausea and vomiting associated with chemotherapy and for appetite stimulation of AIDS patients suffering from anorexia and wasting syndrome.

THC has numerous biological activities, which lend themselves to possible additional therapeutic applications. One potential application is the treatment of glaucoma. Glaucoma leads to progressive damage to the optic nerve through various mechanisms, such as increased pressure (IOP) within the eye caused by decreased blood flow, or poor drainage of fluids, which can lead to vision loss, and is the leading cause of irreversible blindness. Prolonged elevated IOP within the eye that is higher than the tolerance pressure of the retinal ganglion cell (RGC) results in the degeneration of the RGCs. Once RGCs are damaged, they are not regenerated which is why glaucoma is known as a silent disease as there are no major warning signs until vision loss begins. However, studies have shown that topical application of THC had no effect on IOP. Additionally, the American Glaucoma Society has opined that although marijuana can lower the IOP, its side effects and short duration of action, coupled with a lack of evidence that its use alters the course of glaucoma, preclude recommending this drug in any form for the treatment of glaucoma.

Current pharmaceuticals for the treatment of IOP include Latanoprost and Rhopressa®. Although these products do provide relief for the treatment of IOP, side effects of or development of tolerance to current therapies limit the number of treatment options and thus there is still a need for new treatment and therapy to reduce IOP.

### SUMMARY

Described herein is the use of delta-9-tetrahydrocannabinol amino acid ester or derivative thereof and a Rho kinase inhibitor such as, for example, netarsudil or the pharmaceutically acceptable salt thereof for reducing or preventing IOP in a subject in need thereof. The methods involve co-administering to the subject a delta-9-tetrahydrocannabinol amino acid ester or derivative thereof and a Rho kinase inhibitor such as, for example, netarsudil or the pharmaceutically acceptable salt thereof. In one aspect, the delta-9-tetrahydrocannabinol amino acid ester or derivative thereof and the Rho kinase inhibitor can be administered sequentially to the subject. In other aspects, the delta-9-tetrahydrocannabinol amino acid ester or derivative thereof and the Rho kinase inhibitor can be administered to the subject as a single pharmaceutical formulation. The combination of delta-9-tetrahydrocannabinol amino acid ester or derivative thereof and the Rho kinase inhibitor is effective in reducing IOP to a greater extent when compared to independently the delta-9-tetrahydrocannabinol amino acid ester or derivative thereof and the Rho kinase inhibitor.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 shows average IOP (days 1, 3, and 5) vs time profiles in test vs contralateral eyes of DB rabbits after topical administration of THC-VHS-NEC, Rhopressa®, or latanoprost formulations (mean±SD, n=6).

FIG. 2 shows average % drop in IOP (days 1, 3, and 5) vs time profiles in test vs contralateral eyes of DB rabbits after topical administration of THC-VHS-NEC formulation, Rhopressa®, or latanoprost formulations (mean±SD, n=6).

FIG. 3 shows Day 1—IOP vs time profiles in test eye of DB rabbits after topical administration of THC-VHS-NEC, Rhopressa®, or latanoprost formulations (mean±SD, n=6).

FIG. 4 shows Day 1—IOP vs time profiles in contralateral eye of DB rabbits after topical administration of THC-VHS-NEC, Rhopressa®, or latanoprost formulations (mean±SD, n=6).

FIG. 5 shows Day 1—% drop in IOP vs time profiles in test eye of DB rabbits after topical administration of THC-VHS-NEC, Rhopressa®, or latanoprost formulations (mean±SD, n=6).

FIG. 6 shows Day 1—% drop in IOP vs time profiles in contralateral eye of DB rabbits after topical administration of THC-VHS-NEC, Rhopressa®, or latanoprost formulations (mean±SD, n=6).

FIG. 7 shows Day 3—IOP vs time profiles in test eye of DB rabbits after topical administration of THC-VHS-NEC, Rhopressa®, or latanoprost formulations (mean±SD, n=6).

FIG. 8 shows Day 3—IOP vs time profiles in contralateral eye of DB rabbits after topical administration of THC-VHS-NEC, Rhopressa®, or latanoprost formulations (mean±SD, n=6).

FIG. 9 shows Day 3—% drop in IOP vs time profiles in test eye of DB rabbits after topical administration of THC-VHS-NEC, Rhopressa®, or latanoprost formulations (mean±SD, n=6).

FIG. 10 shows Day 3—% drop in IOP vs time profiles in contralateral eye of DB rabbits after topical administration of THC-VHS-NEC, Rhopressa®, or latanoprost formulations (mean±SD, n=6).

FIG. 11 shows Day 5—IOP vs time profiles in test eye of DB rabbits after topical administration of THC-VHS-NEC, Rhopressa®, or latanoprost formulations (mean±SD, n=6).

FIG. 12 shows Day 5—IOP vs time profiles in contralateral eye of DB rabbits after topical administration of THC-VHS-NEC, Rhopressa®, or latanoprost formulations (mean±SD, n=6).

FIG. 13 shows Day 5—% drop in IOP vs time profiles in test eye of DB rabbits after topical administration of THC-VHS-NEC, Rhopressa®, or latanoprost formulations (mean±SD, n=6).

FIG. 14 shows Day 5—% drop in IOP vs time profiles in contralateral eye of DB rabbits after topical administration of THC-VHS-NEC, Rhopressa®, or latanoprost formulations (mean±SD, n=6).

FIG. 15 shows average and individual day (days 1,3 and 5) IOP vs time profiles in test eye of DB rabbits after topical administration of THC-VHS-NEC formulation (mean±SD, n=6).

FIG. 16 shows average and individual day (days 1,3 and 5) IOP vs time profiles in contralateral eye of DB rabbits after topical administration of THC-VHS-NEC formulation (mean±SD, n=6).

FIG. 17 shows average and individual day (days 1,3 and 5) % drop in IOP vs time profiles in test eye of DB rabbits after topical administration of THC-VHS-NEC formulation (mean±SD, n=6).

FIG. 18 shows average and individual day (days 1,3 and 5) % drop in IOP vs time profiles in contralateral eye of DB rabbits after topical administration of THC-VHS-NEC formulation (mean±SD, n=6).

FIG. 19 shows average and individual day (days 1,3 and 5) IOP vs time profiles in test eye of DB rabbits after topical administration of Rhopressa® formulation (mean±SD, n=6).

FIG. 20 shows average and individual day (days 1,3 and 5) IOP vs time profiles in contralateral eye of DB rabbits after topical administration of Rhopressa® formulation (mean±SD, n=6).

FIG. 21 shows average and individual day (days 1,3 and 5) % drop in IOP vs time profiles in test eye of DB rabbits after topical administration of Rhopressa® formulation (mean±SD, n=6).

FIG. 22 shows average and individual day (days 1,3 and 5) % drop in IOP vs time profiles in contralateral eye of DB rabbits after topical administration of Rhopressa® formulation (mean±SD, n=6).

FIG. 23 shows average and individual day (days 1,3 and 5) IOP vs time profiles in test eye of DB rabbits after topical administration of Latanoprost formulation (mean±SD, n=6).

FIG. 24 shows average and individual day (days 1,3 and 5) IOP vs time profiles in contralateral eye of DB rabbits after topical administration of Latanoprost formulation (mean±SD, n=6).

FIG. 25 shows average and individual day (days 1,3 and 5) % drop in IOP vs time profiles in test eye of DB rabbits after topical administration of Latanoprost formulation (mean±SD, n=6).

FIG. 26 shows average and individual day (days 1,3 and 5) % drop in IOP vs time profiles in contralateral eye of DB rabbits after topical administration of Latanoprost formulation (mean±SD, n=6).

FIG. 27 shows average IOP (days 1, 3, and 5) vs time profiles following co-administration of two drugs in contrast to the average IOP (days 1, 3, and 5) vs time profiles obtained following single drug in test eye of DB rabbits after topical administration of THC-VHS-NEC, Rhopressa®, or latanoprost formulations (mean±SD, n=6).

FIG. 28 shows average IOP (days 1, 3, and 5) vs time profiles in test vs contralateral eyes of DB rabbits after topical co-administration of THC-VHS-NEC, Rhopressa®, or latanoprost formulations (mean±SD, n=6).

FIG. 29 shows average % drop in IOP (days 1, 3, and 5) vs time profiles following co-administration of two drugs in contrast to the average IOP (days 1, 3, and 5) vs time profiles obtained following single drug in test eye of DB rabbits after topical administration of THC-VHS-NEC, Rhopressa®, or latanoprost formulations (mean±SD, n=6).

FIG. 30 shows average % drop in IOP (days 1, 3, and 5) vs time profiles following co-administration of two drugs in contrast to the average % drop in IOP (days 1, 3, and 5) vs time profiles obtained following single drug administration in test eye of DB rabbits (mean±SD, n=6).

FIG. 31 shows Day 1—IOP vs time profiles in test eye of DB rabbits after topical co-administration of two formulations (mean±SD, n=6).

FIG. 32 shows Day 1—IOP vs time profiles in contralateral eye of DB rabbits after topical co-administration of two formulations (mean±SD, n=6).

FIG. 33 shows Day 1—% drop in IOP vs time profiles in test eye of DB rabbits after topical co-administration of two formulations (mean±SD, n=6).

FIG. 34 shows Day 1—% drop in IOP vs time profiles in contralateral eye of DB rabbits after topical co-administration of two formulations (mean±SD, n=6).

FIG. 35 shows Day 3—IOP vs time profiles in test eye of DB rabbits after topical co-administration of two formulations (mean±SD, n=6).

FIG. 36 shows Day 3—IOP vs time profiles in contralateral eye of DB rabbits after topical co-administration of two formulations (mean±SD, n=6).

FIG. 37 shows Day 3—% drop in IOP vs time profiles in test eye of DB rabbits after topical co-administration of two formulations (mean±SD, n=6).

FIG. 38 shows Day 3—% drop in IOP vs time profiles in contralateral eye of DB rabbits after topical co-administration of two formulations (mean±SD, n=6).

FIG. 39 shows Day 5—IOP vs time profiles in test eye of DB rabbits after topical co-administration of two formulations (mean±SD, n=6).

FIG. 40 shows Day 5—IOP vs time profiles in contralateral eye of DB rabbits after topical co-administration of two formulations (mean±SD, n=6).

FIG. 41 shows Day 5—% drop in IOP vs time profiles in test eye of DB rabbits after topical co-administration of THC-VHS-NEC, Rhopressa®, & latanoprost formulations (mean±SD, n=6).

FIG. 42 shows Day 5—% drop in IOP vs time profiles in contralateral eye of DB rabbits after topical co-administration of two formulations (mean±SD, n=6).

FIG. 43 shows comparative average IOP (days 1, 3 and 5) vs time profiles following topical administration of Rhopressa® alone, THC-VHS-NEC alone and combination of the two (THC-VHS-NEC+Rhopressa®) in test eye of DB rabbits (mean±SD, n=6).

FIG. 44 shows comparative average IOP (days 1, 3 and 5) vs time profiles against day 1, day 3, day 5 IOP profiles in contralateral eye of DB rabbits after topical co-administration of THC-VHS-NEC+Rhopressa® formulation (mean±SD, n=6).

FIG. 45 shows comparative average % drop in IOP (days 1, 3 and 5) vs time profiles following topical administration of Rhopressa® alone, THC-VHS-NEC alone and combination of the two (THC-VHS-NEC+Rhopressa®) in test eye of DB rabbits (mean±SD, n=6).

FIG. 46 shows comparative average % drop in IOP (days 1, 3 and 5) vs time profiles against day 1, day 3, day 5% drop in IOP profiles in contralateral eye of DB rabbits after topical co-administration of THC-VHS-NEC+Rhopressa® formulation (mean±SD, n=6).

FIG. 47 shows comparative average IOP (days 1, 3 and 5) vs time profiles following topical administration of Rhopressa® alone, Latanoprost alone and combination of the two (Rhopressa® +Latanoprost) in test eye of DB rabbits (mean±SD, n=6).

FIG. 48 shows comparative average IOP (days 1, 3 and 5) vs time profiles against day 1, day 3, day 5 IOP profiles in contralateral eye of DB rabbits after topical co-administration of Rhopressa®+latanoprost formulations (mean±SD, n=6).

FIG. 49 shows comparative average % drop in IOP (days 1, 3 and 5) vs time profiles following topical administration of Rhopressa® alone, Latanoprost alone and combination of the two (Rhopressa®+Latanoprost) in test eye of DB rabbits (mean±SD, n=6).

FIG. 50 shows comparative average % drop in IOP (days 1, 3 and 5) vs time profiles against day 1, day 3, day 5% drop in IOP profiles in contralateral eye of DB rabbits after topical co-administration of Rhopressa®+latanoprost formulations (mean±SD, n=6).

FIG. 51 shows comparative average IOP (days 1, 3 and 5) vs time profiles following topical administration of THC-VHS-NC alone, Latanoprost alone and combination of the two (latanoprost +THC-VHS-NEC) formulations (mean±SD, n=6).

FIG. 52 shows comparative average IOP (days 1, 3 and 5) vs time profiles against day 1, day 3, day 5 IOP profiles in contralateral eye of DB rabbits after topical co-administration of latanoprost+THC-VHS-NEC formulations (mean±SD, n=6).

FIG. 53 shows comparative average % drop in IOP (days 1, 3 and 5) vs time profiles following topical administration of THC-VHS-NC alone, Latanoprost alone and combination of the two (latanoprost+THC-VHS-NEC) formulations (mean±SD, n=6).

FIG. 54 shows comparative average % drop in IOP (days 1, 3 and 5) vs time profiles against day 1, day 3, day 5% drop in IOP profiles in contralateral eye of DB rabbits after topical co-administration of latanoprost+THC-VHS-NEC formulations (mean±SD, n=6).

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by,” “comprising,” “comprises,” “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “an amino acid” includes, but is not limited to, mixtures or combinations of two or more such amino acids, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y.’

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Disclosed are the components to be used to conduct the methods of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

As used herein, the term “admixing” is defined as mixing two or more components together so that there is no chemical reaction or physical interaction. The term “admixing” also includes the chemical reaction or physical interaction between the two or more components.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom, or condition thereof, such as glaucoma. The effect can be therapeutic in terms of a partial or complete cure of a disease, condition, symptom, or adverse effect attributed to the disease, disorder, or condition. The term “treatment” as used herein can include any treatment of glaucoma in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. In one aspect, “treating” and “treatment” includes an improved pharmacological and/or physiological effect when administered a compound described herein when compared to not administering the compound (i.e., the control).

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

As used herein, “effective amount” can refer to the amount of a disclosed compound or pharmaceutical composition provided herein that is sufficient to effect beneficial or desired biological, emotional, medical, or clinical response of a cell, tissue, system, animal, or human. An effective amount can be administered in one or more administrations, applications, or dosages. The term can also include within its scope amounts effective to enhance or restore to substantially normal physiological function.

As used herein, the term “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed.

The term “pharmaceutically acceptable salts,” as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

The term “ophthalmically suitable” describes a material that is not biologically or otherwise undesirable, i.e., without causing an unacceptable level of undesirable biological effects or interacting in a deleterious manner with respect to the eye.

As used herein, “intraocular pressure” (IOP) refers to the fluid pressure of the eye, which is exerted by the aqueous humor of the eye on the surface area of the anterior eye. In one aspect, high intraocular pressure is a risk factor for glaucoma and can result from inflammation, anatomical problems or differences, genetics, medication side effects, and the like. In a further aspect, in a human, normal eye pressure is typically between 10 and 22 mmHg, while IOP in mammals in general typically varies between 8 and 35, with different species having different, but overlapping, ranges.

In one aspect, a “tonometer” is an instrument used to measure IOP in a human or other mammal. A variety of types of tonometers exist. Applanation tonometers are designed based on the assumption that the pressure inside a dry, thin-walled sphere equals the force necessary to flatten its surface divided by the area of flattening, and wherein, in use, the cornea is flattened.

Examples include Goldmann applanation tonometers and Perkins tonometers. Non-contact tonometry also involves flattening the cornea; air puff tonometers and ocular response analyzers use columns of air with increasing intensity. Indentation tonometry is based on the idea that a force will sink into a soft eye further than into a hard eye; examples include Schiotz tonometers, pneumotonometers, and Tono-Pens (which also involve an applanation process). Rebound tonometry involves rebounding a plastic ball on a wire off the eye, where the wire is held in place by an electromagnetic field; IOP is correlated to speed of deceleration of the eye in this device. A Pascal dynamic contour tonometer makes use of a piezoelectric sensor in the tonometer to measure dynamic fluctuations in IOP. Some soft contact lens sensors may be used to measure changes in dimensions of the eye over the course of a day and has been shown to correlate to IOP. In one aspect, numerous tonometers can be used in humans and other mammals to measure IOP. In another aspect, a change in IOP can be measured by taking an initial measurement with a tonometer or other sensor such as described herein, administering a treatment, and taking a second measurement with the same tonometer or other sensor, where a difference between the initial measurement and the second measurement indicates the change in IOP.

Unless otherwise specified, temperatures referred to herein are based on atmospheric pressure (i.e. one atmosphere).

### Methods for Reducing Intraocular Pressure

Described herein are methods for reducing or preventing IOP in a subject. The methods involve administering to the subject a delta-9-tetrahydrocannabinol amino acid ester or derivative thereof and a Rho kinase inhibitor. The combination of delta-9-tetrahydrocannabinol amino acid ester or derivative thereof and the Rho kinase inhibitor is effective in reducing IOP when compared to independently the delta-9-tetrahydrocannabinol amino acid ester or derivative thereof and netarsudil or the pharmaceutically acceptable salt thereof. As demonstrated herein, the co-administration of the delta-9-tetrahydrocannabinol amino acid ester or derivative thereof and the Rho kinase inhibitor can enhance the reduction of IOP initially after administration and provide sustained reduction of IOP when compared to just the administration of the Rho kinase inhibitor. In one aspect, the delta-9-tetrahydrocannabinol amino acid ester has the structure I:

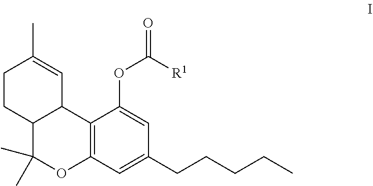

wherein R¹ comprises one or more amino acid residues. In another aspect, the amino acid residue comprises valine, sarcosine, leucine, glutamine, tryptophan, tyrosine, alanine and 4(4-aminophenyl)butyric acid, or a salt thereof, or any combination thereof.

In another aspect, derivatives of the delta-9-tetrahydrocannabinol amino acid ester can be used herein. In one aspect, the delta-9-tetrahydrocannabinol amino acid ester can be reacted with an anhydride or dicarboxylic acid to produce the derivative of the delta-9-tetrahydrocannabinol amino acid ester. In one aspect, the anhydride is succinic anhydride or glutaric anhydride. In another aspect, the dicarboxylic acid is malonic acid, malic acid, glutaric acid, succinic acid, or phthalic acid. In one aspect, the derivative of the delta-9-tetrahydrocannabinol amino acid ester has the structure below, where n is an integer from 1 to 8.

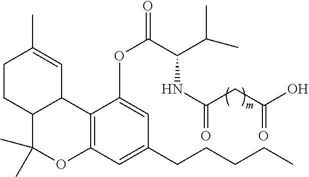

In one aspect, the derivative is delta-9-tetrahydrocannabinol-valine-hemisuccinate, the structure of which is provided below

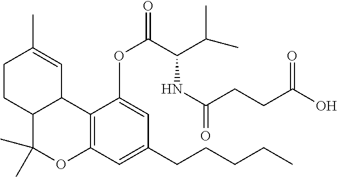 [SMILES: CCCCCc1cc(OC(=O)[C@@H](NC(=O)CCC(=O)O)C(C)C)c2c(c1)OC(C)(C)C1CCC(C)=CC21]

Methods for synthesizing delta-9-tetrahydrocannabinol amino acid esters and derivatives thereof are provided in US Publication No. 2011/0275555, which is incorporated by reference in its entirety.

In one aspect, the disclosed formulations and/or nanoemulsions can include from about 0.01% w/v to about 5% w/v of the delta-9-tetrahydrocannabinol amino acid ester or derivative thereof, or about 0.01%, 0.05%, 0.1%, 0.5%, 1%, 1.5%, 2%, 2.5%, 3%, 3.5%, 4%, 4.5%, or about 5% w/v of the delta-9-tetrahydrocannabinol amino acid ester or derivative thereof, or a combination of any of the foregoing values, or a range encompassing any of the foregoing values.

The delta-9-tetrahydrocannabinol amino acid ester or derivative thereof is formulated as an ophthalmic composition. In one aspect, the delta-9-tetrahydrocannabinol amino acid ester or derivative thereof is formulated as a nanoemulsion. The delta-9-tetrahydrocannabinol amino acid ester or derivative thereof can be formulated with one or more additional components to produce the nanoemulsion.

In one aspect, the nanoemulsion includes an ophthalmically suitable oil. Examples of such oils include, but are not limited to castor oil, cottonseed oil, soybean oil, or sesame oil. In another aspect, the oil is the amount of from about 1% w/v to about 10% w/v of the composition, or about 1% w/v, 1.5% w/v, 2% w/v, 2.5% w/v, 3% w/v, 3.5% w/v, 4% w/v, 4.5% w/v, 5% w/v, 5.5% w/v, 6% w/v, 6.5% w/v, 7% w/v, 7.5% w/v, 8% w/v, 8.5% w/v, 9% w/v, 9.5% w/v, or 10% w/v, where any value can be a lower and upper endpoint of a range (e.g., 1.5% w/v to 4% w/v).

In one aspect, the nanoemulsion includes an ophthalmically suitable nonionic surfactant. In one aspect, the nonionic surfactant is a poloxamer, which is a nonionic triblock copolymer composed of a central hydrophobic chain of polyoxypropylene (e.g., (poly(propylene oxide)) flanked by two hydrophilic chains of polyoxyethylene (e.g., poly(ethylene oxide)). In one aspect, poloxamer has the formula

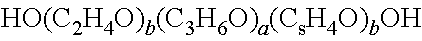

wherein a is from 10 to 100, 20 to 80, 25 to 70, or 25 to 70, or from 50 to 70; b is from 5 to 250, 10 to 225, 20 to 200, 50 to 200, 100 to 200, or 150 to 200. In another aspect, the poloxamer has a molecular weight (MW) from 2,000 to 15,000, 3,000 to 14,000, or 4,000 to 12,000. Poloxamers useful herein are sold under the tradename Pluronic® manufactured by BASF. Non-limiting examples of poloxamers useful herein include, but are not limited to, those in the table below. In one aspect, the poloxamer is F-407 (Pluronic® F-127). Useful poloxamers are presented in Table 1:

TABLE 1
Exemplary Poloxamers Useful Herein

| | | Average number | Average number | |
|---|---|---|---|---|
| Copolymer | MW | of EO units | of PO units | CMC (M) |
| F68 | 8,400 | 152.73 | 28.97 | 4.8 × 10⁻⁴ |
| P103 | 4,950 | 33.75 | 59.74 | 6.1 × 10⁻⁶ |
| P105 | 6,500 | 73.86 | 56.03 | 6.2 × 10⁻⁶ |
| P123 | 5,750 | 39.2 | 69.4 | 4.4 × 10⁻⁶ |
| F127 | 12,600 | 200.45 | 65.17 | 2.8 × 10⁻⁶ |
| L121 | 4,400 | 10.00 | 68.28 | 1.1 × 10⁻⁶ |

In another aspect, the nonionic surfactant is a polysorbate. Polysorbates are oily liquids derived from ethoxylated sorbitan (a derivative of sorbitol) esterified with fatty acids. Examples of polysorbates include polysorbate 20, 40, 60, or 80.

In one aspect, the nonionic surfactant includes a combination of a poloxamer and polysorbate. In one aspect, the poloxamer is the amount of from about 0.01% w/v to about 1% w/v of the composition, or about 0.01% w/v, 0.05% w/v, 0.1% w/v, 0.2% w/v, 0.3% w/v, 0.4% w/v, 0.5% w/v, 0.6% w/v, 0.7% w/v, 0.8% w/v, 0.9% w/v, or 1% w/v, where any value can be a lower and upper endpoint of a range (e.g., 0.2% w/v to 0.4% w/v). In another aspect, the polysorbate is the amount of from about 0.5% w/v to about 5% w/v of the composition, or about 1% w/v, 1.5% w/v, 2% w/v, 2.5% w/v, 3% w/v, 3.5% w/v, 4% w/v, 4.5% w/v, or 5% w/v, where any value can be a lower and upper endpoint of a range (e.g., 1.5% w/v to 4% w/v).

In one aspect, the nanoemulsion includes an ophthalmically suitable polymer to modify certain properties of the nanoemulsion. In one aspect, the polymer is a crosslinked polyacrylic acid such as, for Example, Carbopol 940 manufactured by Lubrizol. In one aspect, the polymer is the amount of from about 0.1% w/v to about 2% w/v of the composition, or about 0.1% w/v, 0.2% w/v, 0.4% w/v, 0.6% w/v, 0.8% w/v, 1.0% w/v, 1.2% w/v, 1.4% w/v, 1.6% w/v, 1.8% w/v, or 2.0% w/v, where any value can be a lower and upper endpoint of a range (e.g., 0.4% w/v to 1.2% w/v).

In one aspect, the nanoemulsion includes an ophthalmically suitable polyol, which is a compound having two or more hydroxyl groups. In one aspect, the polyol is glycerin. In one aspect, the polyol is in the amount of from about 1% w/v to about 5% w/v of the composition, or about 1% w/v, 1.5% w/v, 2% w/v, 2.5% w/v, 3% w/v, 3.5% w/v, 4% w/v, 4.5% w/v, or 5% w/v, where any value can be a lower and upper endpoint of a range (e.g., 1.5% w/v to 4% w/v).

In one aspect, the nanoemulsion includes an ophthalmically suitable ethoxylated tocopherol or tocotrienol. In one aspect, the ethoxylated tocopherol or tocotrienol is D-alpha-tocopherol polyethylene glycol. In one aspect the nanoemulsion includes Vitamin E polyethoxylated succinate (TPGS). In one aspect, the ethoxylated tocopherol or tocotrienol is in the amount of from about 0.0001% w/v to about 0.01% w/v of the composition, or about 0.0001% w/v, 0.0005% w/v, 0.001% w/v, 0.002% w/v, 0.003% w/v, 0.004% w/v, 0.005% w/v, 0.006% w/v, 0.007% w/v, 0.008% w/v, 0.009% w/v, 0.01% w/v, where any value can be a lower and upper endpoint of a range (e.g., 0.001% w/v to 0.007% w/v).

In one aspect, the nanoemulsion is composed of a delta-9-tetrahydrocannabinol amino acid ester or derivative thereof, an oil, a poloxamer, a polysorbate, a crosslinked polyacrylic acid, a polyol, an ethoxylated tocopherol or tocotrienol, and water.

In another aspect, the nanoemulsion can include from about 0.01% w/v to about 2% w/v of a delta-9-tetrahydrocannabinol amino acid ester or derivative thereof, from about 1% to about 10% w/v of an ophthalmically suitable oil, from about 0.51% w/v to about 6% w/v of an ophthalmically suitable surfactant, from about 0.1% w/v to about 2% w/v of an ophthalmically suitable polymer, from about 1% to about 5% w/v of an ophthalmically suitable polyol, from about 0.0001% w/v to about 0.01% w/v of an ophthalmically suitable ethoxylated tocopherol or tocotrienol, and water.

In some aspects, the formulations and/or nanoemulsions can further include the Rho kinase inhibitor. In one aspect, the Rho kinase inhibitor can be netarsudil or a pharmaceutically acceptable salt thereof. In one aspect, the formulations can include from about 0.005% w/v to about 0.05% w/v of the Rho kinase inhibitor, or about 0.005%, 0.01%, 0.015%, 0.2%, 0.25%, 0.3%, 0.35%, 0.4%, 0.45%, or about 0.5% of the Rho kinase inhibitor, or a combination of any of the foregoing values, or a range encompassing any of the foregoing values.

In one aspect, the nanoemulsion can be produced by ultrasonication. For example, the delta-9-tetrahydrocannabinol amino acid ester or derivative thereof is admixed with the oil and subsequently heated to produce a hot lipid phase. The poloxamer, polysorbate, and polyol are admixed in water and heated to produce a hot aqueous phase. The hot aqueous phase is added to the heated lipid phase under constant mixing to form a coarse emulsion. The coarse emulsion is then homogenized at, for example, 11,000 rpm for 5 min at 65° C. using T 25 digital Ultra-Turrax (IKA, Germany) to form a fine emulsion. The fine emulsion was allowed to slowly cool before being placed in an ice bath and subjected to ultra-sonication (SONICS® Vibra-Cell™, Newtown, CT, USA) using a 3-mm stepped microtip probe (40% amplitude; pulse on: 10 s, pulse off: 15 s; time: 10 min).

The physical properties of the nanoemulsion can be modified and fine-tuned as needed. In one aspect, the nanoemulsion has an average droplet size (z-average) of from about 200 nm to about 250 nm as measured by dynamic light scattering (e.g., Zetasizer Nano ZS Zen3600), or about 200 nm, 205 nm, 210 nm, 215 nm, 220 nm, 225 nm, 230 nm, 235 nm, 240 nm, 245 nm, or 250 nm, where any value can be a lower and upper endpoint of a range (e.g., 210 nm to 240 nm).

In one aspect, the nanoemulsion has a polydispersity index of from about 0.15 to about 0.25 as measured by dynamic light scattering (e.g., Zetasizer Nano ZS Zen3600), or about 0.15, 0.16, 0.17, 0.18, 0.15, 0.19, 0.20, 0.21, 0.22, 0.23, 0.24, or 0.25, where any value can be a lower and upper endpoint of a range (e.g., 0.18 to 0.23).

In one aspect, the nanoemulsion has a zeta potential of from about −20 mV to about −60 mV as measured by dynamic light scattering (e.g., Zetasizer Nano ZS Zen3600), or about −20 mV, −25 mV, −30 mV, −35 mV, −40 mV, −45 mV, −50 mV, −55 mV, or −60 mV, where any value can be a lower and upper endpoint of a range (e.g., −25 mV to −35 mV).

In certain aspects, the nanoemulsion can be sterilized prior to administration. In one aspect, the nanoemulsion can be filtered. In one aspect, the nanoemulsion can be filtered through a micrometer filter membrane (e.g., a 0.22-μm filter). In another aspect, the nanoemulsion can be moist heat sterilized. The methods described herein also involve the co-administration of a Rho kinase inhibitor. In one aspect, the Rho kinase inhibitor is AT-13148, BA-210, B-Elemene DJ4, Fasudil, GSK-576371, GSK429286A, H-1152, hydroxyfasudil, LX-7101, RKI-1447, ripasudil, TCS-7001, thiazovivin, verosudil Y-30141, Y-33075, or Y-39983. In another aspect, the rho kinase inhibitor is netarsudil or the pharmaceutically acceptable salt thereof.

In one aspect, netarsudil mesylate, which is also referred to as the commercially-available ophthalmic solution Rhopressa®, can be used herein. In one aspect, ophthalmic compositions of the netarsudil or the pharmaceutically acceptable salt thereof can be formulated with ophthalmically suitable buffers and excipients. In one aspect, netarsudil or the pharmaceutically acceptable salt thereof is formulated as an ophthalmic composition having a concentration of about 0.005% w/v to about 0.05% w/v, or about 0.005% w/v, 0.010% w/v, 0.015% w/v, 0.020% w/v, 0.025% w/v, 0.030% w/v, 0.035% w/v, 0.040% w/v, 0.045% w/v, or 0.050% w/v, where any value can be a lower and upper endpoint of a range (e.g., 0.010% w/v to 0.030% w/v). In one aspect, the ophthalmic composition includes netarsudil or the pharmaceutically acceptable salt thereof at a concentration of 0.02% w/v.

In one aspect a nanoemulsion comprising the Rho Kinase inhibitor and the delta-9-tetrahydrocannabinol amino acid ester or derivative thereof, can be formulated wherein both the Rho Kinase inhibitor, or its lipophilic derivatives, as well as the delta-9-tetrahydrocannabinol amino acid ester or derivative thereof, is dissolved in the lipid phase of the emulsion. In another aspect the Rho Kinase inhibitor or its salts is dissolved in the aqueous phase of the emulsion, whereas the delta-9-tetrahydrocannabinol amino acid ester or derivative thereof is dissolved in the lipid phase of the emulsion. Additional excipients, such as solubilizers, surfactants, buffering agents, tonicity adjusting agents, permeation enhancers, mucoadhesive agents, viscosity enhancers, emulsion stabilizers may be added at concentrations relevant for ophthalmic formulations.

The delta-9-tetrahydrocannabinol amino acid ester or derivative thereof and the Rho kinase inhibitor can be administered topically to the eye of the subject in need of treatment or prevention of IOP. Methods for topical administration include eye droppers and other suitable devices for applying eye drops to the surface of the eye. The order in which the delta-9-tetrahydrocannabinol amino acid ester or derivative thereof and the netarsudil or the pharmaceutically acceptable salt thereof can be administered can vary. In one aspect, the delta-9-tetrahydrocannabinol amino acid ester or derivative thereof is administered prior to the administration of the Rho kinase inhibitor. In another aspect, the delta-9-tetrahydrocannabinol amino acid ester or derivative thereof is administered after the administration of the Rho kinase inhibitor. In another aspect, the delta-9-tetrahydrocannabinol amino acid ester or derivative thereof is administered concurrently with the administration of the Rho kinase inhibitor. In one aspect, the delta-9-tetrahydrocannabinol amino acid ester or derivative thereof and the Rho kinase inhibitor can be formulated in a single pharmaceutical formulation.

Depending upon the condition of the subject, the delta-9-tetrahydrocannabinol amino acid ester or derivative thereof and the Rho kinase inhibitor can each be administered multiple times over a specified period of time. For example, the delta-9-tetrahydrocannabinol amino acid ester or derivative thereof and the Rho kinase inhibitor can be administered every day, every two days, every three days, or every five days. In another aspect, the delta-9-tetrahydrocannabinol amino acid ester or derivative thereof and the Rho kinase inhibitor can be administered once a day or twice a day. The amount and duration of the administration of the delta-9-tetrahydrocannabinol amino acid ester or derivative thereof and the Rho kinase inhibitor can be varied depending upon the symptoms of the subject. In one aspect, the delta-9-tetrahydrocannabinol amino acid ester or derivative thereof are administered once per day for at least five days, or once per day for 10 or more days.

Also disclosed herein are methods for treating or reducing IOP in a subject, the methods including at least the step of administering the disclosed formulations and/or nanoemulsions to the subject. In one aspect, performing the method can result in a decrease in IOP in the subject of from about 15% to about 35% compared to IOP in the subject prior to performing the method, or of about 15, 20, 25, 30, or about 35%, or a combination of any of the foregoing values, or a range encompassing any of the foregoing values. In another aspect, after performing the method, the decrease in IOP remains at least about 15%. In another aspect, performing the method can result in a decrease in IOP in the subject of from about 5 to about 10 mm Hg compared to the IOP in the subject prior to performing the method, or of about 5, 6, 7, 8, 9, or about 10 mm Hg, or a combination of any of the foregoing values, or a range encompassing any of the foregoing values. In one aspect, the maximum decrease in IOP relative to IOP prior to performing the method occurs from about 4 to about 7 hours after performing the method, or at about 4, 5, 6, or about 7 hours, or a combination of any of the foregoing values, or a range encompassing any of the foregoing values. In one aspect, a decrease in IOP occurs in the eye in which the delta-9-tetrahydrocannabinol amino acid ester or derivative thereof and the Rho kinase inhibitor are administered, or in the contralateral eye, or both.

As demonstrated in the Examples, the combination of delta-9-tetrahydrocannabinol amino acid ester or derivative thereof and the Rho kinase inhibitor is effective in reducing IOP when compared to independently the delta-9-tetrahydrocannabinol amino acid ester or derivative thereof and the Rho kinase inhibitor. In one aspect, the combination of delta-9-tetrahydrocannabinol amino acid ester or derivative thereof and the Rho kinase inhibitor can increase the reduction of IOP by up to 40%, up to 50%, or up 60% when compared to use of only the delta-9-tetrahydrocannabinol amino acid ester or derivative thereof and the Rho kinase inhibitor.

### Aspects

The present disclosure can be described in accordance with the following numbered aspects, which should not be confused with the claims.

Aspect 1. A method for treating or preventing elevated intraocular pressure (IOP) in a subject in need thereof, the method comprising administering to the subject a delta-9-tetrahydrocannabinol amino acid ester or derivative thereof and a Rho kinase inhibitor.

Aspect 2. The method of aspect 1, wherein the Rho kinase inhibitor is netarsudil or a pharmaceutically acceptable salt thereof.

Aspect 3. The method of aspect 1 or 2, wherein the delta-9-tetrahydrocannabinol amino acid ester has the structure I:

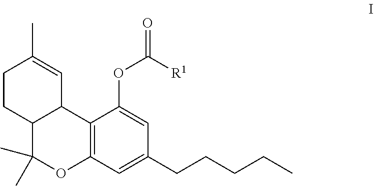

wherein R¹ comprises valine, sarcosine, leucine, glutamine, tryptophan, tyrosine, alanine, 4(4-aminophenyl)butyric acid, or a salt thereof.

Aspect 4. The method of aspect 1 or 2, wherein the derivative of the delta-9-tetrahydrocannabinol amino acid ester has the structure II

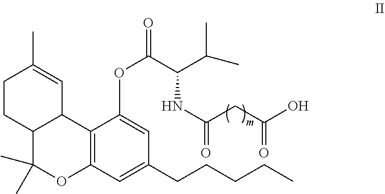

where n is an integer from 1 to 9.

Aspect 5. The method of aspect 1 or 2, wherein the derivative of the delta-9-tetrahydrocannabinol amino acid ester is delta-9-tetrahydrocannabinol-valine-hemisuccinate.

Aspect 6. The method of any one of aspects 1-5, wherein the delta-9-tetrahydrocannabinol amino acid ester or derivative thereof comprises a component of a nanoemulsion.

Aspect 7. The method of any one of aspects 1-6, wherein the delta-9-tetrahydrocannabinol amino acid ester or derivative thereof and the Rho kinase inhibitor are each administered topically to an eye of the subject.

Aspect 8. The method of any one of aspects 1-7, wherein the method is performed once per day for at least five days.

Aspect 9. A nanoemulsion comprising a delta-9-tetrahydrocannabinol amino acid ester or derivative thereof, an ophthalmically suitable oil, an ophthalmically suitable surfactant, and water.

Aspect 10. The nanoemulsion of aspect 9, wherein the delta-9-tetrahydrocannabinol amino acid ester has the structure I:

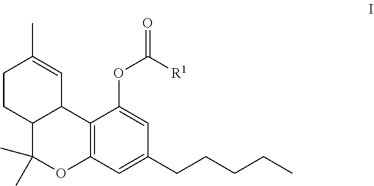

wherein R¹ comprises valine, sarcosine, leucine, glutamine, tryptophan, tyrosine, alanine, 4(4-aminophenyl)butyric acid, or a salt thereof.

Aspect 11. The nanoemulsion of aspect 9, wherein the derivative of the delta-9-tetrahydrocannabinol amino acid ester has the structure II

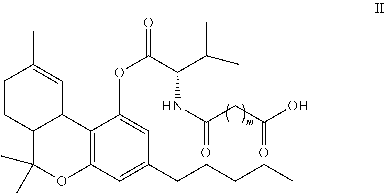

where n is an integer from 1 to 9.

Aspect 12. The nanoemulsion of aspect 9 or 10, wherein the derivative of the delta-9-tetrahydrocannabinol amino acid ester is delta-9-tetrahydrocannabinol-valine-hemisuccinate.

Aspect 13. The nanoemulsion of any one of aspects 9-12, wherein the nanoemulsion comprises from about 0.01% w/v to about 2% w/v of the delta-9-tetrahydrocannabinol amino acid ester or derivative thereof.

Aspect 14. The nanoemulsion of any one of aspects 9-13, wherein the ophthalmically suitable oil comprises castor oil, cottonseed oil, soybean oil, sesame oil, or any combination thereof.

Aspect 15. The nanoemulsion of any one of aspects 9-14, wherein the nanoemulsion comprises from about 1% to about 10% w/v of the ophthalmically suitable oil.

Aspect 16. The nanoemulsion of any one of aspects 9-15, wherein the ophthalmically suitable surfactant comprises a nonionic surfactant.

Aspect 17. The nanoemulsion of any one of aspects 9-16, wherein the ophthalmically suitable surfactant comprises a poloxamer, a polysorbate, or any combination thereof.

Aspect 18. The nanoemulsion of aspect 17, wherein the nanoemulsion comprises from about 0.01% w/v to about 1% w/v of the poloxamer and from about 0.5% w/v to about 5% w/v of the polysorbate.

Aspect 19. The nanoemulsion of any one of aspects 9-18, further comprising an ophthalmically suitable polymer.

Aspect 20. The nanoemulsion of aspect 19, wherein the ophthalmically suitable polymer comprises a crosslinked polyacrylic acid.

Aspect 21. The nanoemulsion of any one of aspects 9-20, further comprising an ophthalmically suitable polyol.

Aspect 22. The nanoemulsion of aspect 21, wherein the ophthalmically suitable polyol comprises glycerin.

Aspect 23. The nanoemulsion of any one of aspects 9-22, further comprising an ophthalmically suitable ethoxylated tocopherol or tocotrienol.

Aspect 24. The nanoemulsion of aspect 23, wherein the ophthalmically suitable ethoxylated tocopherol or tocotrienol comprises D-alpha-tocopherol polyethylene glycol, vitamin E polyethoxylated succinate, or any combination thereof.

Aspect 25. The nanoemulsion of aspect 23 or 24, wherein the nanoemulsion comprises from about 0.0001% w/v to about 0.01% w/v of the ophthalmically suitable ethoxylated tocopherol or tocotrienol.

Aspect 26. A nanoemulsion comprising from about 0.01% w/v to about 2% w/v of a delta-9-tetrahydrocannabinol amino acid ester or derivative thereof, from about 1% to about 10% w/v of an ophthalmically suitable oil, from about 0.51% w/v to about 6% w/v of an ophthalmically suitable surfactant, from about 0.1% w/v to about 2% w/v of an ophthalmically suitable polymer, from about 1% to about 5% w/v of an ophthalmically suitable polyol, from about 0.0001% w/v to about 0.01% w/v of an ophthalmically suitable ethoxylated tocopherol or tocotrienol, and water.

Aspect 27. The nanoemulsion of any one of aspects 9-26, further comprising a Rho kinase inhibitor.

Aspect 28. The nanoemulsion of aspect 27, wherein the Rho kinase inhibitor is netarsudil or a pharmaceutically acceptable salt thereof.

Aspect 29. The nanoemulsion of aspect 27 or 28, wherein the nanoemulsion comprises from about 0.005% w/v to about 0.05% w/v of the Rho kinase inhibitor.

Aspect 30. The nanoemulsion of any one of aspects 9-29, having an average droplet size of from about 200 nm to about 250 nm.

Aspect 31. The nanoemulsion of any one of aspects 9-30, having a polydispersity index of from about 0.15 to about 0.25.

Aspect 32. The nanoemulsion of any one of aspects 9-31, having a zeta potential of from about −20 mV to about −60 mV.

Aspect 33. A method for treating or preventing elevated IOP, the method comprising administering the nanoemulsion of any one of aspects 9-32 to a subject.

### Examples

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Developmental Summary

The current set of studies were undertaken to compare the IOP lowering effect of Latanoprost, and Netarsudil (Rhopressa®) formulations (Table 3) and THC-VHS-NEC formulation (Tables 4 and 5) in 28 week old, male pigmented (Dutch Belted; DB) rabbits, with an average weight of 2.0+0.3 kg. The rabbits were split into three groups. Each group received a single formulation (THC-VHS-NEC, Latanoprost or Rhopressa®) for the first five days and then a combination of two formulations for the next five days. The second formulation was administered 15 minutes after the administration of the first formulation. Treatment protocols are summarized in Table 2:

TABLE 2
Treatment Groups^{a}

| | | | | |
|---|---|---|---|---|
| | Volume | | | Dosage |
| Group | (μL) | Formulation | Concentration | Frequency |

Days 1-5

| | | | | |
|---|---|---|---|---|
| 1 | 50 | THC-VHS-NEC | 1.0% | 1 × day |
| 2 | 50 | Latanoprost | 0.005% | 1 × day |
| 3 | 50 | Rhopressa ® | 0.02% | 1 × day |

Days 6-10

| | | | | |
|---|---|---|---|---|
| 1 | 50 | THC-VHS-NEC | 1.0% | 1 × day |
| | 50 | Rhopressa ® | 0.02% | 1 × day |
| 2 | 50 | Latanoprost | 0.005% | 1 × day |
| | 50 | THC-VHS-NEC | 1.0% | 1 × day |
| 3 | 50 | Rhopressa ® | 0.02% | 1 × day |
| | 50 | Latanoprost | 0.005% | 1 × day |

^{a}n = 6 for all groups and treatments.

IOP was measured on Days 1, 3 and 5 while the rabbits were receiving a single formulation and then on Days 6, 8 and 10 while on the combination treatment. Administration of all treatments was topical (50 μL) and once daily, with the left eye being treated and the contralateral eye (right eye) remaining untreated.

TABLE 3
Commercial Samples Used as Comparators

| Name of | Active | | | Batch | Lot | Exp. |
|---|---|---|---|---|---|---|
| product | ingredient | Company | Concentration | number | number | date |
| Latanoprost | Latanoprost | Bausch + | 0.005% (125 | NDC - | 346471 | April |
| ophthalmic | | Lomb | mcg/2.5 mL) | 24208- | | 2023 |
| solution | | | | 463-25 | | |
| (0.005%) | | | | | | |
| Rhopressa | Netarsudil | Aerie | 0.02% (0.5 | NDC - | 97429 | December |
| (0.02%) | mesylate | | mg/2.5 mL) | 60758- | | 2022 |
| | | | | 802-05 | | |

TABLE 4
Composition of THC-VHS-NEC^{a}

| | |
|---|---|
| Ingredients | Amount (% w/v) |

Lipid phase

| | |
|---|---|
| Sesame oil | 5 |
| THC-VHS | 1 |

Aqueous phase

| | |
|---|---|
| Vitamin E TPGS | 0.002 |
| Poloxamer 407 | 0.2 |
| Tween 80 | 2.0 |
| Glycerin | 2.25 |
| Carbopol 940 | 0.4 |
| Water | QS |

^{a}Batch size: 10 mL, batch number Se-5-117

TABLE 5
Physical and Chemical Characteristics
of THC-VHS-NEC Formulation^{a}

| | | | |
|---|---|---|---|
| Droplet size (mm) | PDI | ZP (mV) | Assay (%) |

Before autoclave

| | | | |
|---|---|---|---|
| 222.3 ± 2.9 | 0.16 ± 0.01 | −395 ± 0.7 | 103.8 ± 0.7 |

After autoclave

| | | | |
|---|---|---|---|
| 221.4 ± 6.2 | 0.19 ± 0.05 | −40.3 ± 0.5 | 98.8 ± 0.8 |

^{a}Batch size: 10 mL, batch number Se-5-117

### Single Treatment Multiple Day Application Studies

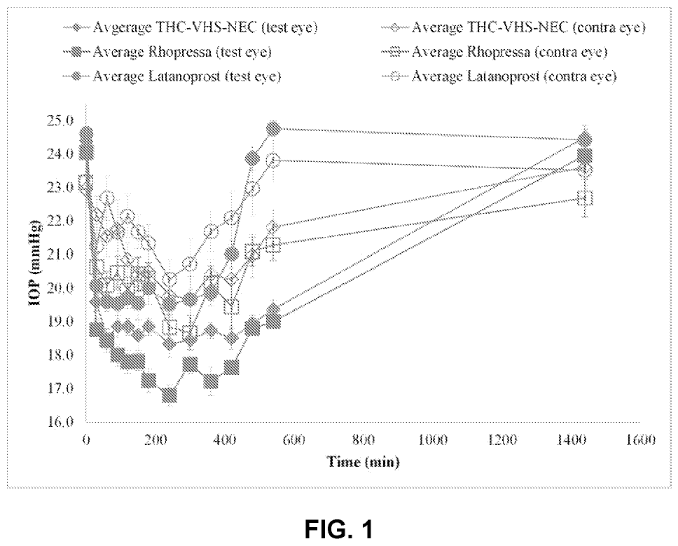

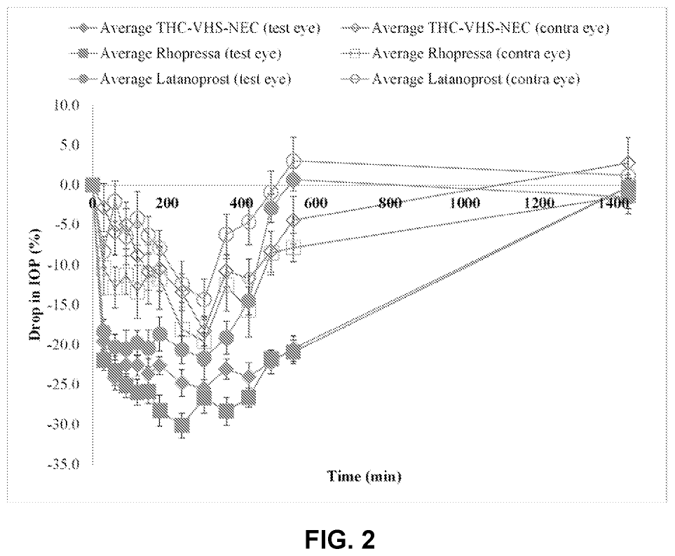

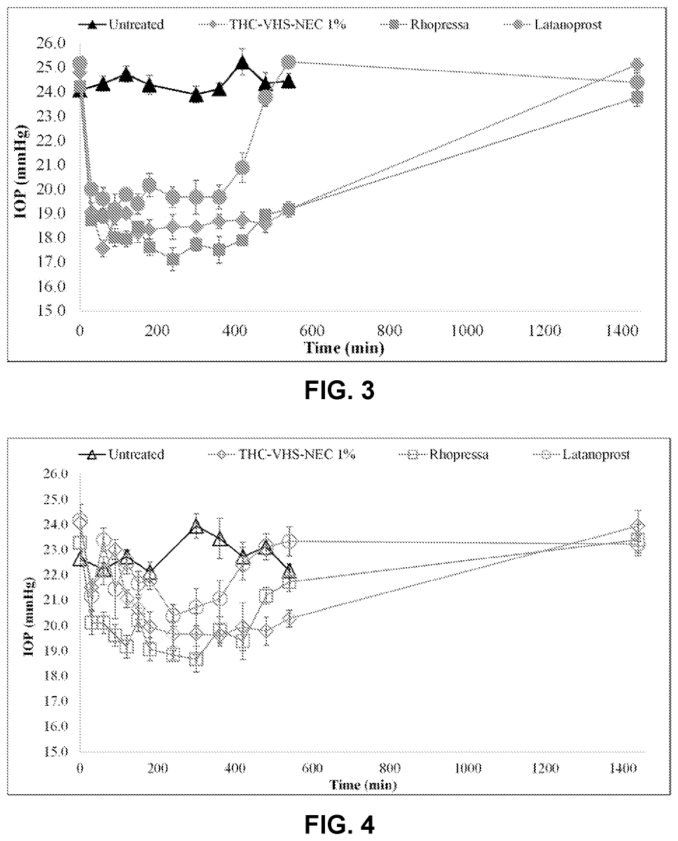

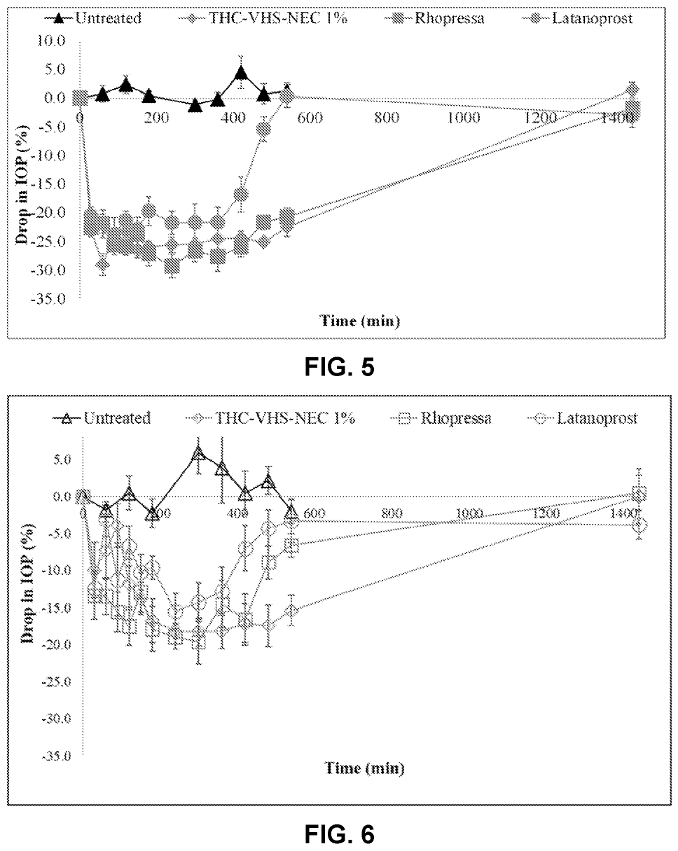

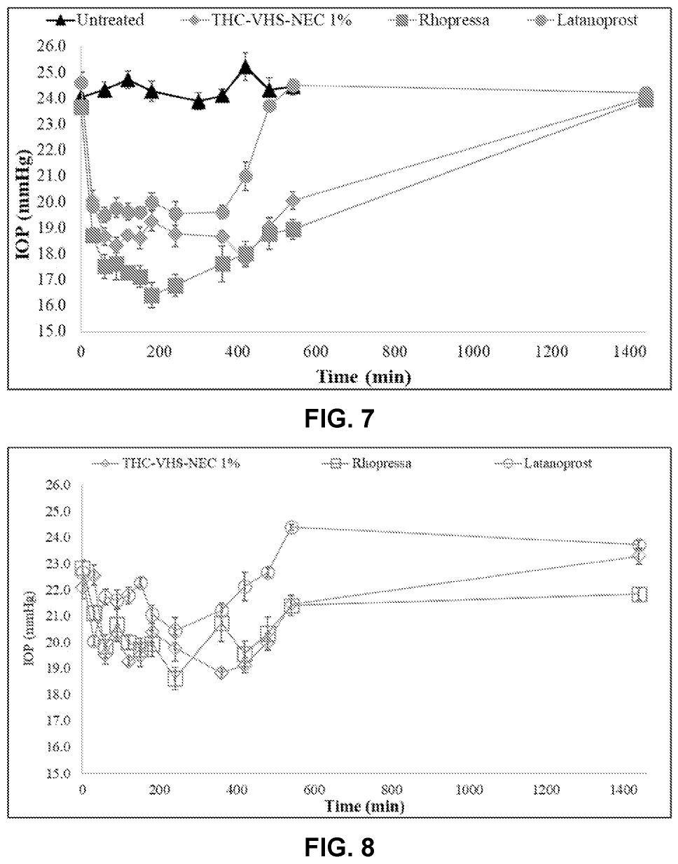

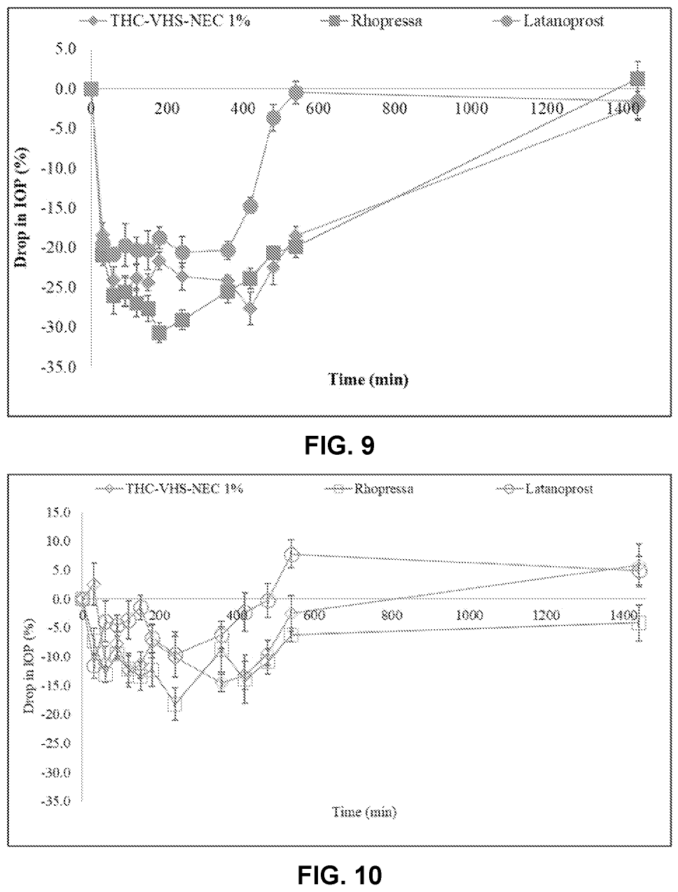

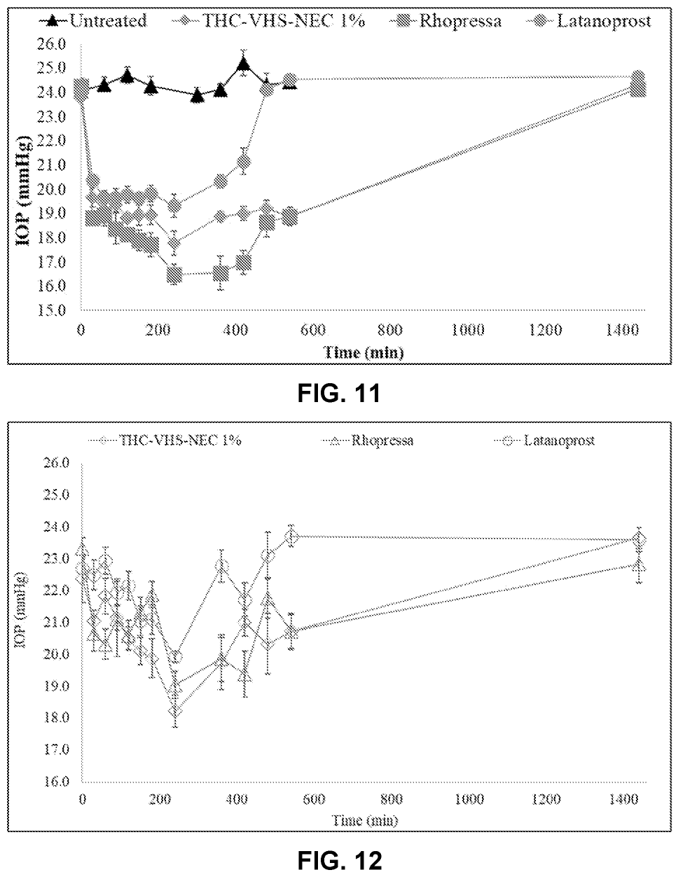

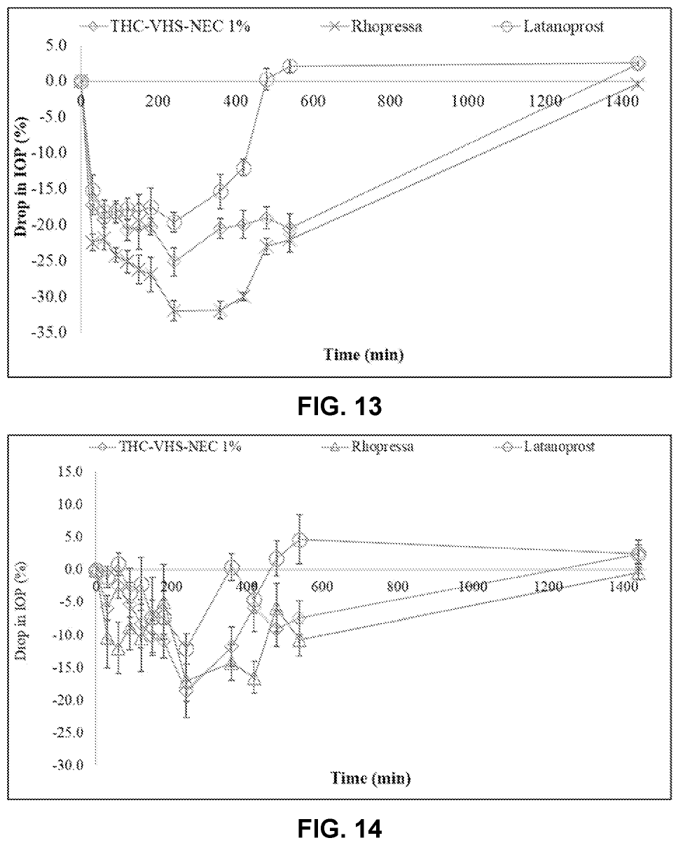

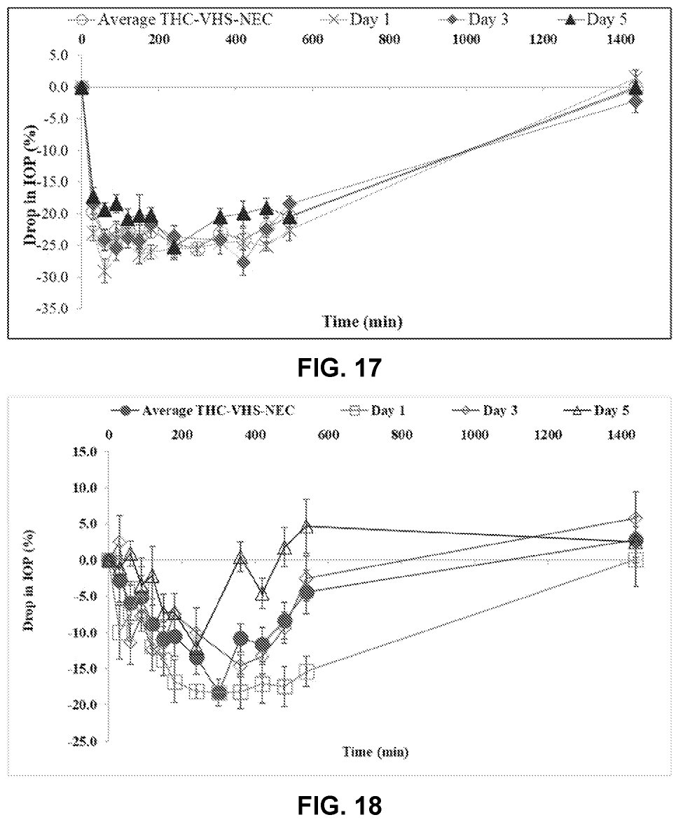

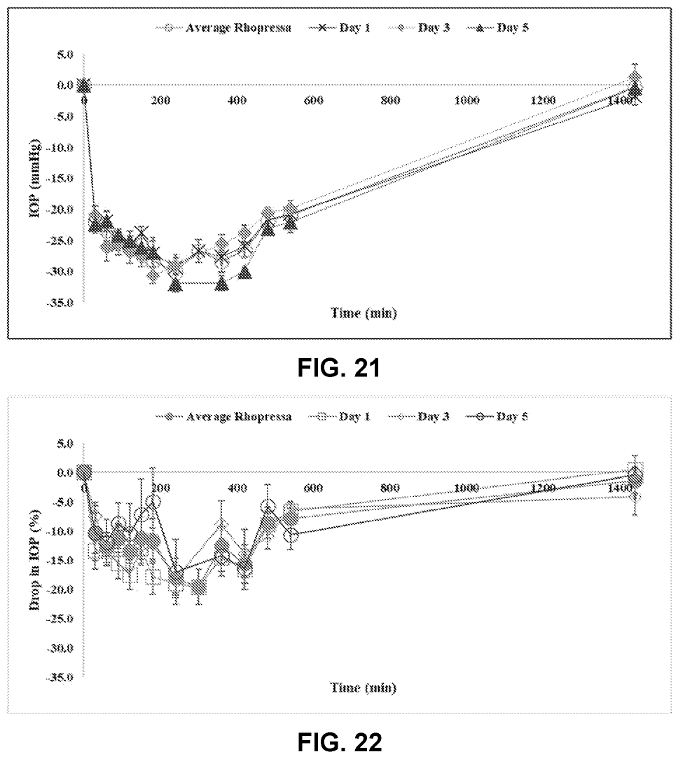

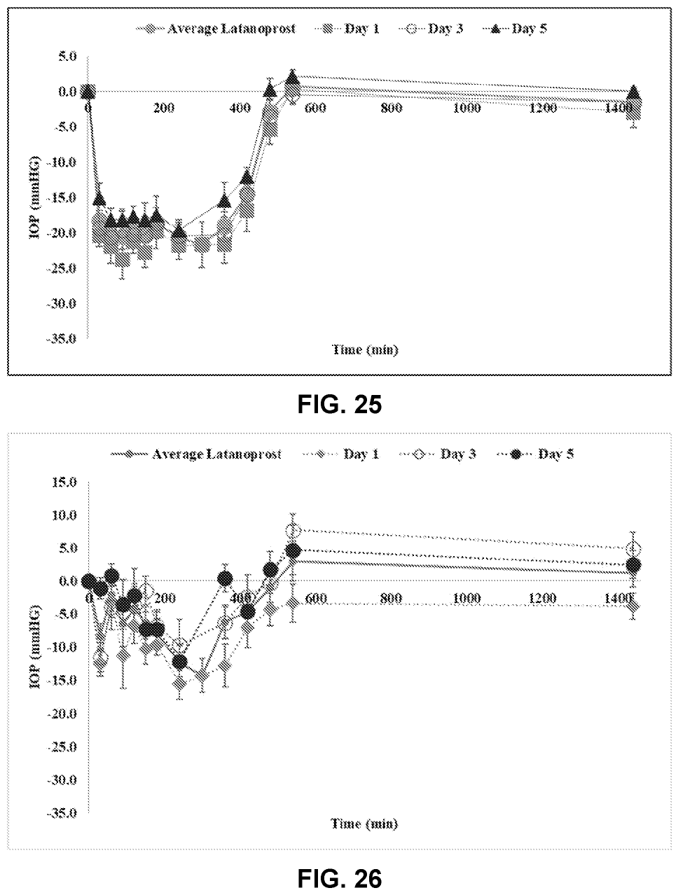

The IOP profile following multi-day (QD, 5 days) exposure to a single formulation, in pigmented DB rabbits, is presented in FIGS. 1-26. Results include the following:

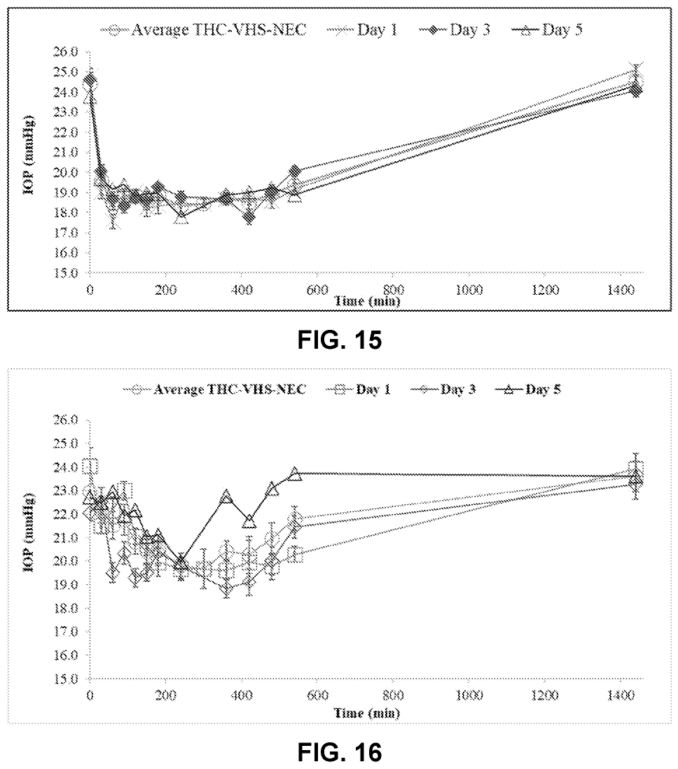

THC-VHS-NEC (FIGS. 15-18) formulation demonstrated an average max drop in IOP of about 27.5+2.1% and maintained an average drop of 21.1+1.6% from 30 to 540 minutes. IOP returned to baseline within 24 h. The time to max drop in IOP varied—60, 420, 240 minutes (day 1, 3, 5)—and the average max IOP drop decreased over the 5 days from 7.2 to 6.0 mmHg. See also Table 6 below.

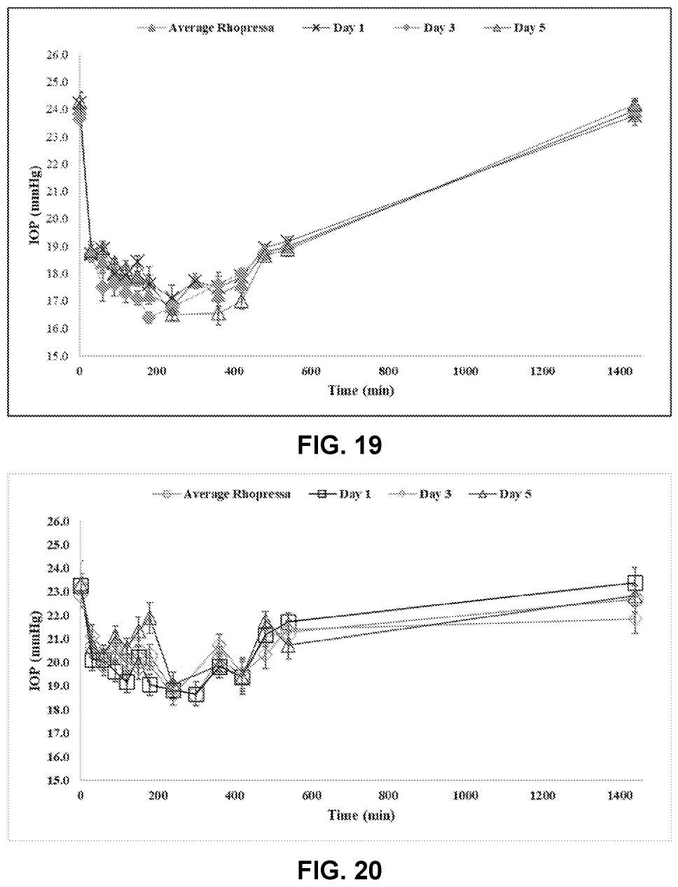

Rhopressa® (FIGS. 19-22) demonstrated an average max drop in IOP of about 30.6+1.4% and maintained an average drop of 25.4+2.9% from 30 to 540 minutes. IOP returned to baseline within 24 h. The max drop in IOP occurred between 180-240 minutes. See also Table 7 below.

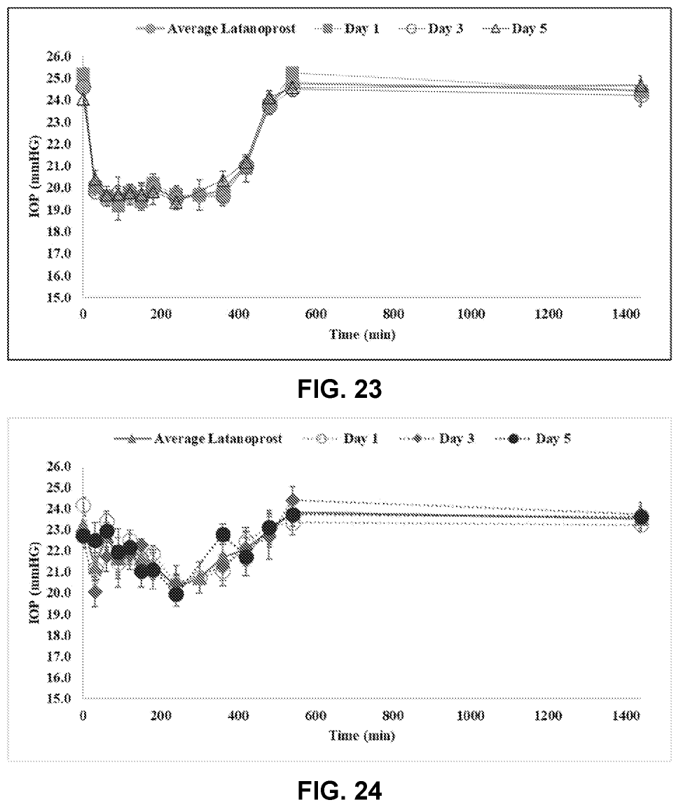

Latanoprost (FIGS. 23-26) demonstrated an average max drop in IOP of about 21.3+2.1% and maintained an average drop of 19.4+1.9% from 30 to 420 mins. IOP returned to baseline within 480 mins. The max drop in IOP occurred between 60, 90 and 240 mins on days 1, 3 and 5. The max IOP decreased over the 5 days from 6.0 to 4.7 mmHg. See also Table 8 below.

TABLE 6
THC-VHS-NEC IOP and % Drop in IOP
Average for THC-VHS-NEC

| Min | 0 | 30 | 60 | 90 | 120 | 150 | 180 | 240 | 300 | 360 | 420 | 480 | 540 | 1440 |
|---|---|---|---|---|---|---|---|---|---|---|---|---|---|---|
| Test eye | 24.4 | 19.6 | 18.5 | 18.9 | 18.9 | 18.6 | 18.9 | 18.3 | 18.4 | 18.7 | 18.5 | 18.9 | 19.4 | 24.5 |
| SEM | 0.4 | 0.3 | 0.3 | 0.3 | 0.2 | 0.5 | 0.3 | 0.4 | 0.2 | 0.2 | 0.3 | 0.3 | 0.3 | 0.3 |
| Drop in IOP | 0.0 | −19.6 | −24.1 | −22.6 | −22.6 | −23.7 | −22.6 | −24.8 | −25.5 | −23.0 | −24.0 | −22.2 | −20.5 | −0.5 |
| SEM | 0.0 | 1.4 | 1.5 | 1.4 | 1.3 | 1.9 | 1.1 | 1.7 | 1.1 | 1.3 | 1.7 | 1.5 | 1.6 | 0.9 |

Day 1

| Min | 0 | 30 | 60 | 90 | 120 | 150 | 180 | 240 | 300 | 360 | 420 | 480 | 540 | 1440 |
|---|---|---|---|---|---|---|---|---|---|---|---|---|---|---|
| Test eye | 24.8 | 19.1 | 17.6 | 18.8 | 19.0 | 18.2 | 18.3 | 18.4 | 18.4 | 18.7 | 18.7 | 18.6 | 19.2 | 25.1 |
| SEM | 0.4 | 0.4 | 0.3 | 0.3 | 0.2 | 0.4 | 0.4 | 0.5 | 0.2 | 0.3 | 0.3 | 0.3 | 0.3 | 0.2 |
| Drop in IOP | 0.0 | −23.1 | −29.0 | −24.0 | −23.3 | −26.4 | −26.0 | −25.6 | −25.5 | −24.5 | −24.4 | −25.1 | −22.5 | 1.4 |
| SEM | 0.0 | 1.1 | 1.9 | 1.0 | 0.9 | 1.4 | 1.1 | 1.3 | 1.1 | 1.8 | 1.2 | 0.7 | 1.6 | 1.3 |

Day 3

| Min | 0 | 30 | 60 | 90 | 120 | 150 | 180 | 240 | 360 | 420 | 480 | 540 | 1440 |
|---|---|---|---|---|---|---|---|---|---|---|---|---|---|
| Test eye | 24.6 | 20.1 | 18.7 | 18.3 | 18.7 | 18.6 | 19.3 | 18.8 | 18.7 | 17.8 | 19.1 | 20.1 | 24.1 |
| SEM | 0.4 | 0.3 | 0.4 | 0.4 | 0.2 | 0.3 | 0.2 | 0.3 | 0.2 | 0.4 | 0.3 | 0.3 | 0.3 |
| Drop in IOP | 0.0 | −18.4 | −24.1 | −25.4 | −23.8 | −24.3 | −21.6 | −23.6 | −24.1 | −27.7 | −22.4 | −18.4 | −2.8 |
| SEM | 0.0 | 1.6 | 1.7 | 1.9 | 1.6 | 1.1 | 1.1 | 1.7 | 0.7 | 2.1 | 2.2 | 1.2 | 1.4 |

Day 5

| Min | 0 | 30 | 60 | 90 | 120 | 150 | 180 | 240 | 360 | 420 | 480 | 540 | 1440 |
|---|---|---|---|---|---|---|---|---|---|---|---|---|---|
| Test eye | 23.8 | 19.7 | 19.2 | 19.4 | 18.8 | 18.9 | 18.9 | 17.8 | 18.9 | 19.0 | 19.2 | 18.9 | 24.3 |
| SEM | 0.4 | 0.2 | 0.2 | 0.3 | 0.3 | 0.6 | 0.2 | 0.5 | 0.3 | 0.3 | 0.1 | 0.3 | 0.2 |
| Drop in IOP | 0.0 | −17.2 | −19.3 | −18.4 | −20.7 | −20.2 | −20.2 | −25.2 | −20.5 | −20.0 | −19.0 | −20.4 | 0.0 |
| SEM | 0.0 | 1.4 | 1.1 | 1.4 | 1.5 | 3.2 | 1.2 | 2.0 | 1.4 | 1.9 | 1.5 | 2.0 | 0.0 |

TABLE 7
Rhopressa ® IOP and % Drop in IOP
Average for Rhopressa ®

| Min | 0 | 30 | 60 | 90 | 120 | 150 | 180 | 240 | 300 | 360 | 420 | 480 | 540 | 1440 |
|---|---|---|---|---|---|---|---|---|---|---|---|---|---|---|
| Test eye | 24.1 | 18.8 | 18.4 | 18.0 | 17.8 | 17.8 | 17.2 | 16.8 | 17.7 | 17.2 | 17.6 | 18.8 | 19.0 | 24.0 |
| SEM | 0.3 | 0.3 | 0.4 | 0.3 | 0.3 | 0.3 | 0.4 | 0.3 | 0.3 | 0.4 | 0.2 | 0.1 | 0.2 | 0.3 |
| Drop in IOP | 0.0 | −22.0 | −23.3 | −25.1 | −25.9 | −25.9 | −28.2 | −30.1 | −26.7 | −28.3 | −26.6 | −21.8 | −20.9 | −0.9 |
| SEM | 0.0 | 1.3 | 1.8 | 1.5 | 1.6 | 1.5 | 1.9 | 1.6 | 1.9 | 1.7 | 1.2 | 1.1 | 1.5 | 1.0 |

Day 1

| Min | 0 | 30 | 60 | 90 | 120 | 150 | 180 | 240 | 300 | 360 | 420 | 480 | 540 | 1440 |
|---|---|---|---|---|---|---|---|---|---|---|---|---|---|---|
| Test eye | 24.2 | 18.7 | 18.9 | 18.0 | 17.9 | 18.4 | 17.6 | 17.1 | 17.7 | 17.5 | 17.9 | 18.9 | 19.2 | 23.8 |
| SEM | 0.4 | 0.2 | 0.3 | 0.4 | 0.3 | 0.2 | 0.3 | 0.5 | 0.3 | 0.5 | 0.2 | 0.1 | 0.2 | 0.4 |
| Drop in IOP | 0.0 | −22.6 | −21.9 | −25.6 | −25.8 | −23.8 | −27.1 | −29.3 | −26.7 | −27.6 | −26.0 | −21.7 | −20.7 | −1.8 |
| SEM | 0.0 | 1.3 | 1.6 | 1.7 | 1.5 | 1.0 | 2.1 | 2.0 | 1.9 | 2.6 | 1.7 | 1.3 | 1.5 | 1.5 |

Day 3

| Min | 0 | 30 | 60 | 90 | 120 | 150 | 180 | 240 | 360 | 420 | 480 | 540 | 1440 |
|---|---|---|---|---|---|---|---|---|---|---|---|---|---|
| Test eye | 23.7 | 18.7 | 17.5 | 17.6 | 17.3 | 17.1 | 16.4 | 16.8 | 17.6 | 18.0 | 18.8 | 18.9 | 23.9 |
| SEM | 0.3 | 0.3 | 0.5 | 0.4 | 0.3 | 0.2 | 0.2 | 0.3 | 0.3 | 0.2 | 0.2 | 0.2 | 0.4 |
| Drop in IOP | 0.0 | −20.9 | −26.0 | −25.6 | −26.9 | −27.6 | −30.7 | −29.1 | −25.5 | −23.9 | −20.6 | −19.9 | −1.1 |
| SEM | 0.0 | 1.4 | 2.3 | 1.8 | 1.8 | 1.6 | 1.3 | 1.2 | 1.4 | 1.3 | 0.9 | 1.3 | 1.5 |

Day 5

| Min | 0 | 30 | 60 | 90 | 120 | 150 | 180 | 240 | 360 | 420 | 480 | 540 | 1440 |
|---|---|---|---|---|---|---|---|---|---|---|---|---|---|
| Test eye | 24.3 | 18.8 | 18.9 | 18.4 | 18.2 | 17.9 | 17.7 | 16.5 | 16.6 | 17.0 | 18.7 | 18.9 | 24.2 |
| SEM | 0.3 | 0.3 | 0.2 | 0.2 | 0.3 | 0.4 | 0.5 | 0.2 | 0.4 | 0.3 | 0.2 | 0.3 | 0.2 |
| Drop in IOP | 0.0 | −22.4 | −21.9 | −24.2 | −25.1 | −26.2 | −26.9 | −32.0 | −31.9 | −30.0 | −23.0 | −22.1 | 0.0 |
| SEM | 0.0 | 1.1 | 1.6 | 1.0 | 1.6 | 2.0 | 2.4 | 1.4 | 1.2 | 0.6 | 1.1 | 1.7 | 0.0 |

TABLE 8
Latanoprost IOP and % Drop in IOP
Average for Latanoprost

| Min | 0 | 30 | 60 | 90 | 120 | 150 | 180 | 240 | 300 | 360 | 420 | 480 | 540 | 1440 |
|---|---|---|---|---|---|---|---|---|---|---|---|---|---|---|
| Test eye | 24.6 | 20.1 | 19.6 | 19.5 | 19.7 | 19.6 | 20.0 | 19.5 | 19.7 | 19.9 | 21.0 | 23.9 | 24.8 | 24.4 |
| SEM | 0.2 | 0.3 | 0.3 | 0.5 | 0.3 | 0.5 | 0.5 | 0.4 | 0.7 | 0.4 | 0.4 | 0.4 | 0.2 | 0.4 |
| Drop in IOP | 0.0 | −18.3 | −20.3 | −20.5 | −19.8 | −20.5 | −18.7 | −20.6 | −21.7 | −19.1 | −14.5 | −2.9 | 0.7 | −2.2 |
| SEM | 0.0 | 1.5 | 1.6 | 2.4 | 1.6 | 2.3 | 2.2 | 1.8 | 3.2 | 2.1 | 1.7 | 1.8 | 1.4 | 1.6 |

Day 1

| Min | 0 | 30 | 60 | 90 | 120 | 150 | 180 | 240 | 300 | 360 | 420 | 480 | 540 | 1440 |
|---|---|---|---|---|---|---|---|---|---|---|---|---|---|---|
| Test Eye | 25.2 | 20.0 | 19.6 | 19.2 | 19.8 | 19.4 | 20.2 | 19.7 | 19.7 | 19.7 | 20.9 | 23.8 | 25.2 | 24.4 |
| SEM | 0.3 | 0.3 | 0.4 | 0.6 | 0.3 | 0.4 | 0.5 | 0.4 | 0.7 | 0.5 | 0.6 | 0.4 | 0.2 | 0.4 |
| Drop in IOP | 0.0 | −20.5 | −21.9 | −23.7 | −21.3 | −22.9 | −19.7 | −21.8 | −21.7 | −21.7 | −16.8 | −5.4 | 0.3 | −3.0 |
| SEM | 0.0 | 1.5 | 2.4 | 2.9 | 1.6 | 2.1 | 2.5 | 2.0 | 3.2 | 2.6 | 3.1 | 2.1 | 2.0 | 2.2 |

Day 3

| Min | 0 | 30 | 60 | 90 | 120 | 150 | 180 | 240 | 360 | 420 | 480 | 540 | 1440 |
|---|---|---|---|---|---|---|---|---|---|---|---|---|---|
| Test eye | 24.6 | 19.8 | 19.5 | 19.8 | 19.6 | 19.6 | 20.0 | 19.6 | 19.6 | 21.0 | 23.7 | 24.5 | 24.2 |
| SEM | 0.1 | 0.2 | 0.1 | 0.7 | 0.4 | 0.6 | 0.3 | 0.4 | 0.3 | 0.2 | 0.4 | 0.3 | 0.5 |
| Drop in IOP | 0.0 | −19.4 | −20.7 | −19.6 | −20.3 | −20.3 | −18.7 | −20.5 | −20.3 | −14.7 | −3.6 | −0.4 | −3.6 |
| SEM | 0.0 | 0.9 | 0.8 | 2.7 | 1.7 | 2.4 | 1.3 | 1.9 | 1.2 | 1.0 | 1.7 | 1.4 | 2.5 |

Day 5

| Min | 0.00 | 30 | 60 | 90 | 120 | 150 | 180 | 240 | 360 | 420 | 480 | 540 | 1440 |
|---|---|---|---|---|---|---|---|---|---|---|---|---|---|
| Test eye | 24.1 | 20.4 | 19.7 | 19.7 | 19.8 | 19.7 | 19.8 | 19.3 | 20.3 | 21.2 | 24.1 | 24.6 | 24.7 |
| SEM | 0.2 | 0.4 | 0.2 | 0.3 | 0.4 | 0.6 | 0.6 | 0.3 | 0.4 | 0.3 | 0.3 | 0.1 | 0.4 |
| Drop in IOP | 0.0 | −15.2 | −18.2 | −18.2 | −17.8 | −18.2 | −17.5 | −19.6 | −15.4 | −12.0 | 0.3 | 2.1 | 0.0 |
| SEM | 0.0 | 2.1 | 1.6 | 1.5 | 1.5 | 2.4 | 2.6 | 1.4 | 2.4 | 1.2 | 1.5 | 0.9 | 0.0 |

Rhopressa® was superior to THC-VHS-NEC formulation with regards to the max drop in IOP. However, both exhibited a duration of action of at least 9 hours—IOP remained about 20% below baseline even at 9 hours after administration. Both Rhopressa® and THC-VHS-NEC formulations performed better than Latanoprost in terms of both max drop in IOP as well as duration of activity. All formulations produced a corresponding drop in IOP in the contralateral eye. No irritation or redness (visual observation) was observed in any of the rabbit eyes across the various treatment groups. Results are summarized in Table 9:

TABLE 9
Max Drop in IOP and Max % Drop in
IOP for Single Drug Formulations

| | | Day 1 | Day 3 | Day 5 |
|---|---|---|---|---|
| THC-VHS-NEC | Max IOP drop | 7.2 | 6.9 | 6 |
| | (mmHg) | 60 min | 420 min | 240 min |
| | Max IOP drop (%) | −29.2 | −28.1 | −25.2 |
| | | 60 min | 420 min | 240 min |
| Rhopressa | Max IOP drop | 7.1 | 7.3 | 7.8 |
| | (mmHg) | 240 min | 180 min | 240 min |
| | Max IOP drop (%) | −29.3 | −30.7 | −32 |
| | | 240 min | 180 min | 240 min |
| Latanoprost | Max IOP drop | 6 | 5.1 | 4.7 |
| | (mmHg) | 90 min | 60 min | 240 min |
| | Max IOP drop (%) | −23.7 | −20.7 | −19.6 |

| | | |
|---|---|---|
| 90 min | 60 min | 240 min |

### Combination Dose Multiple Day Application Studies

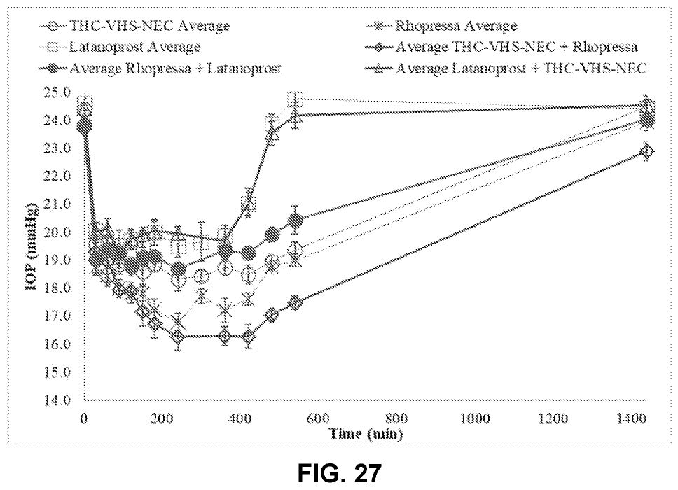

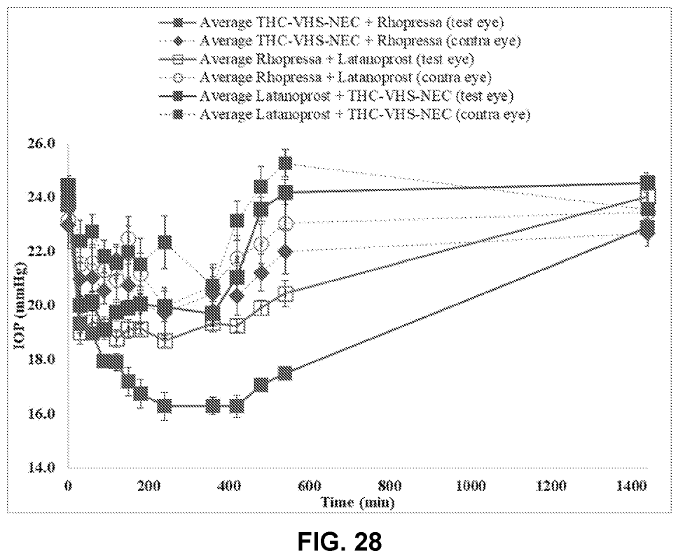

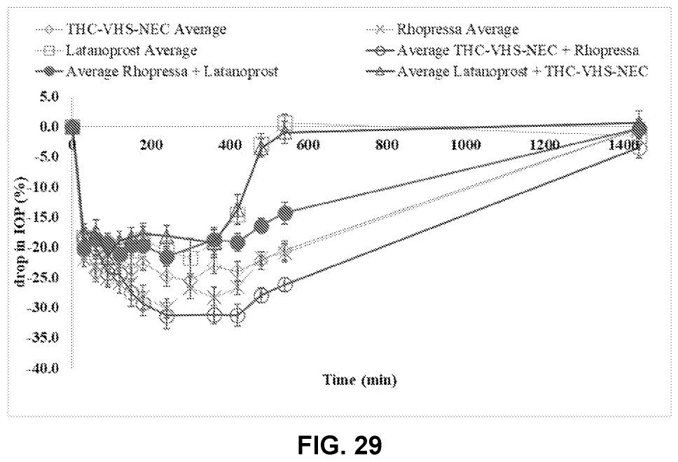

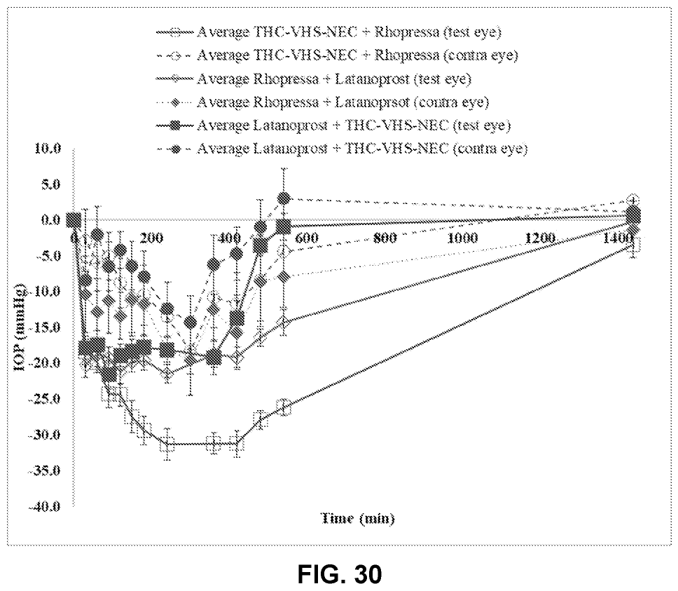

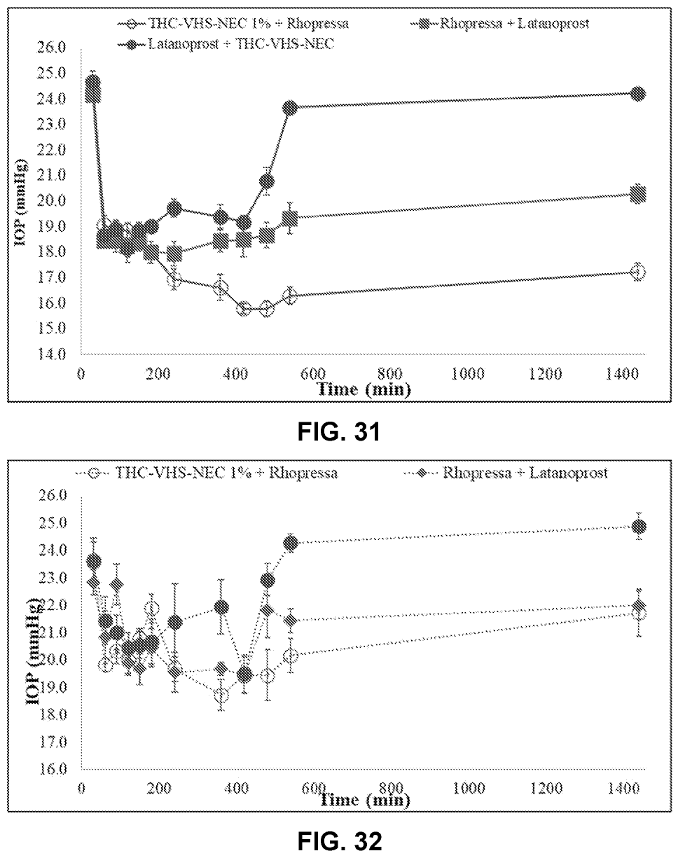

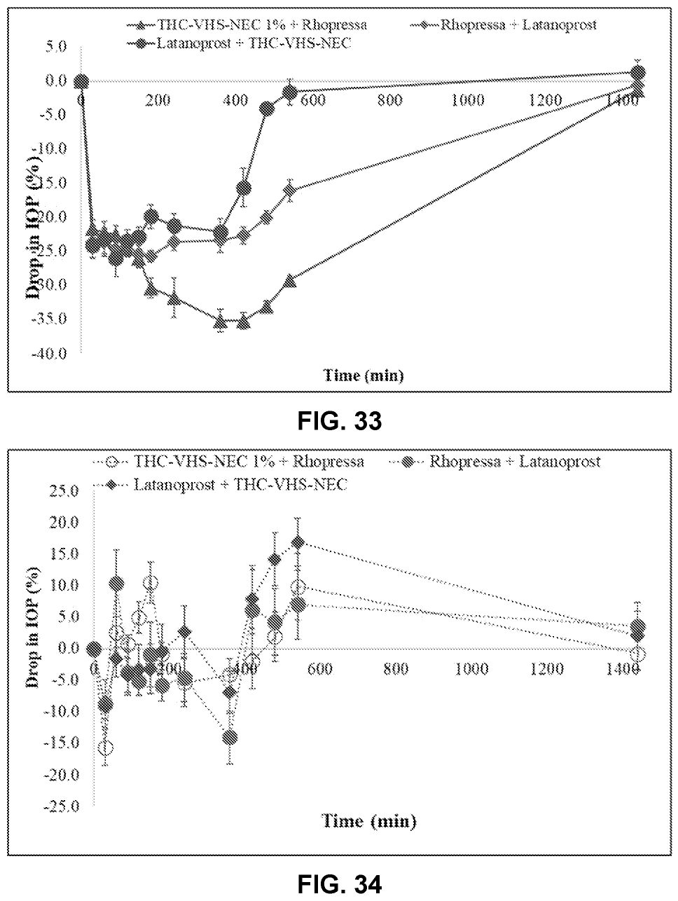

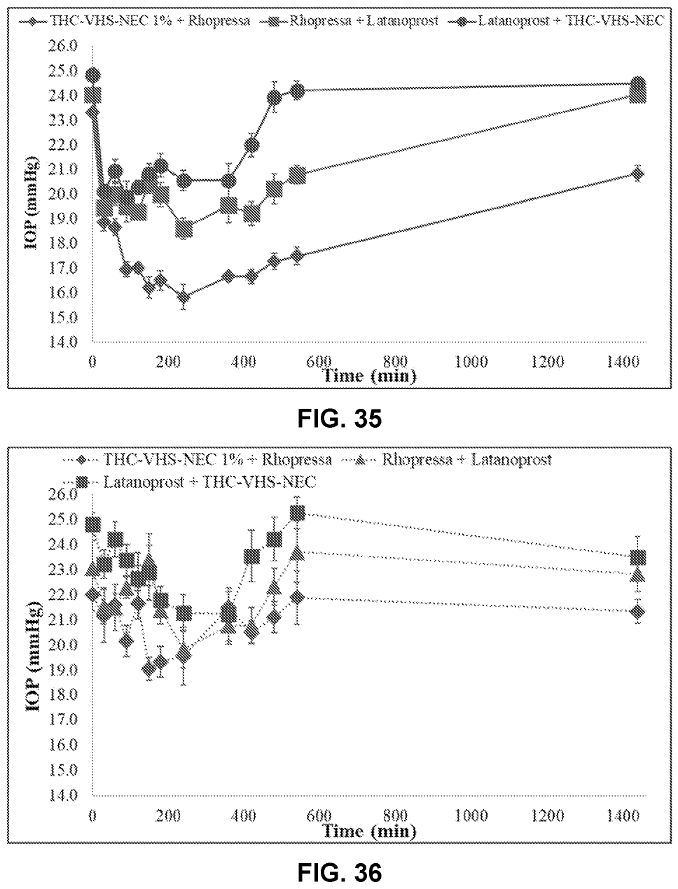

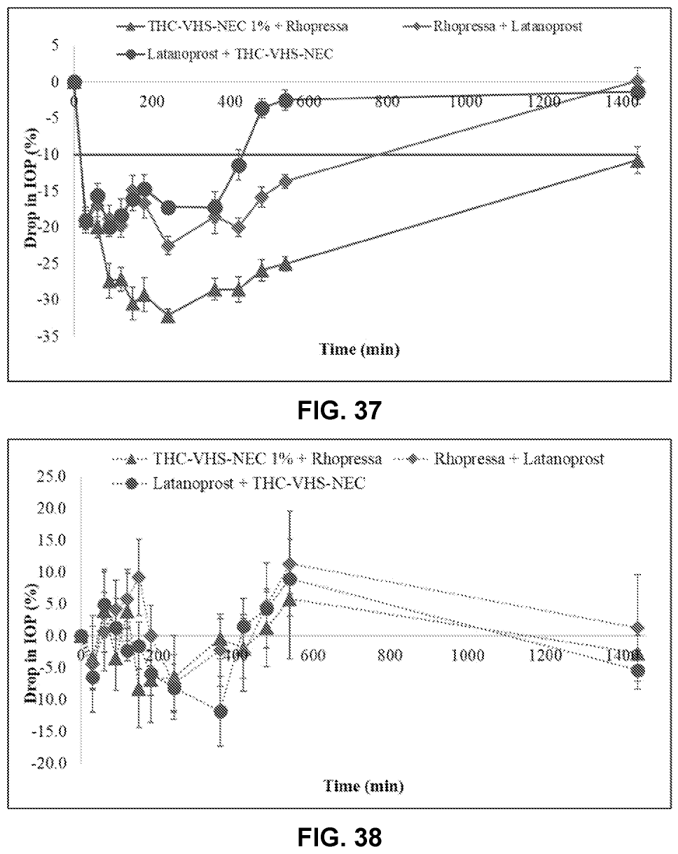

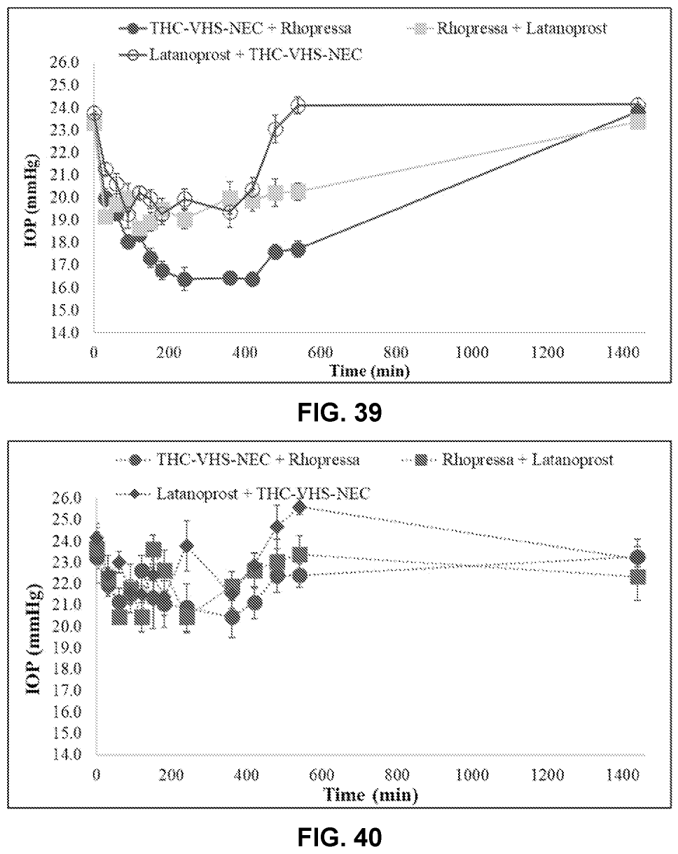

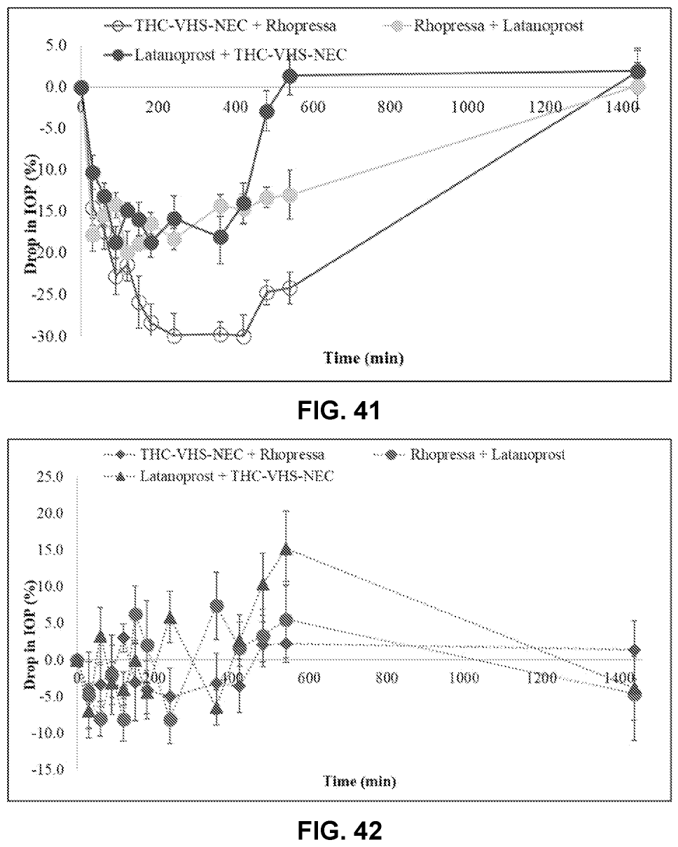

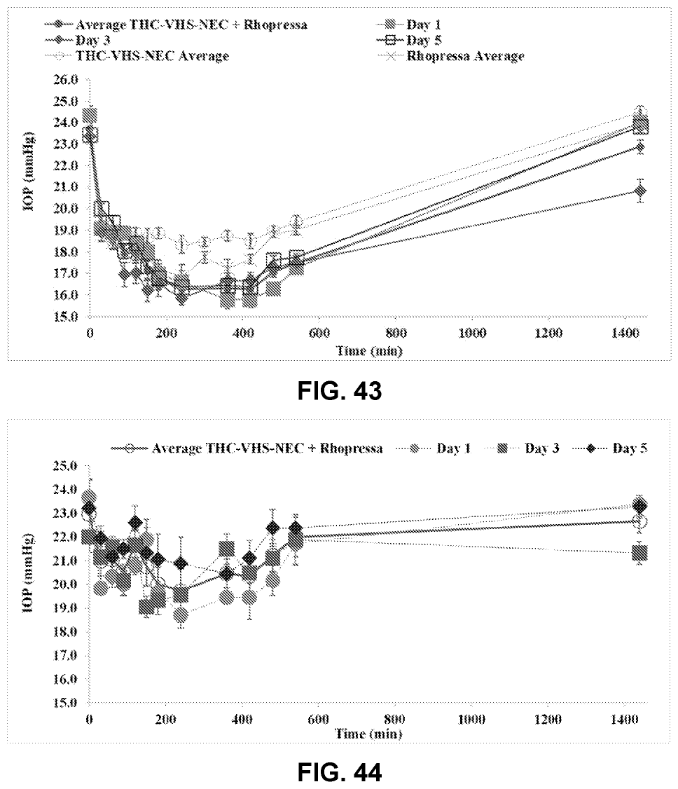

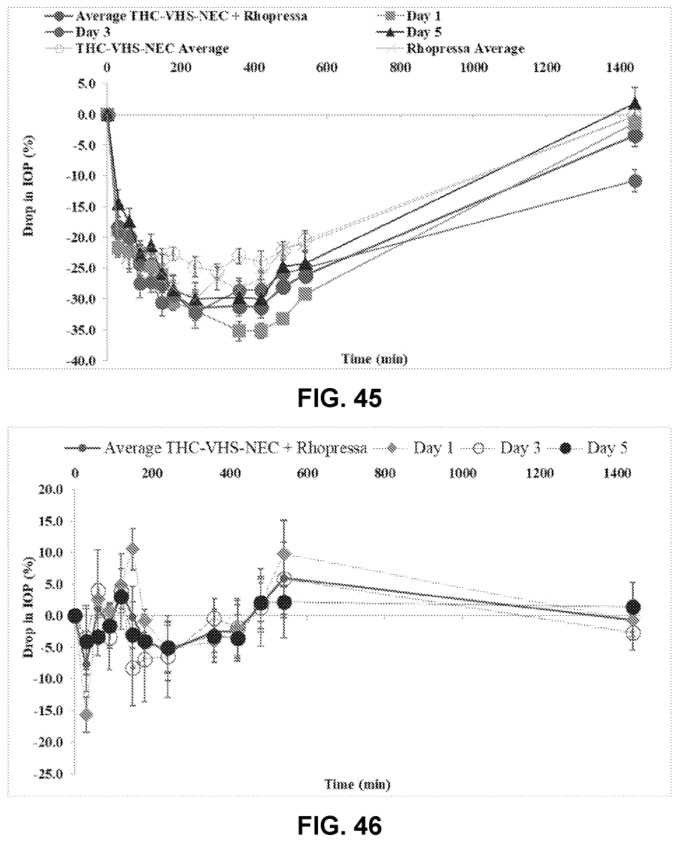

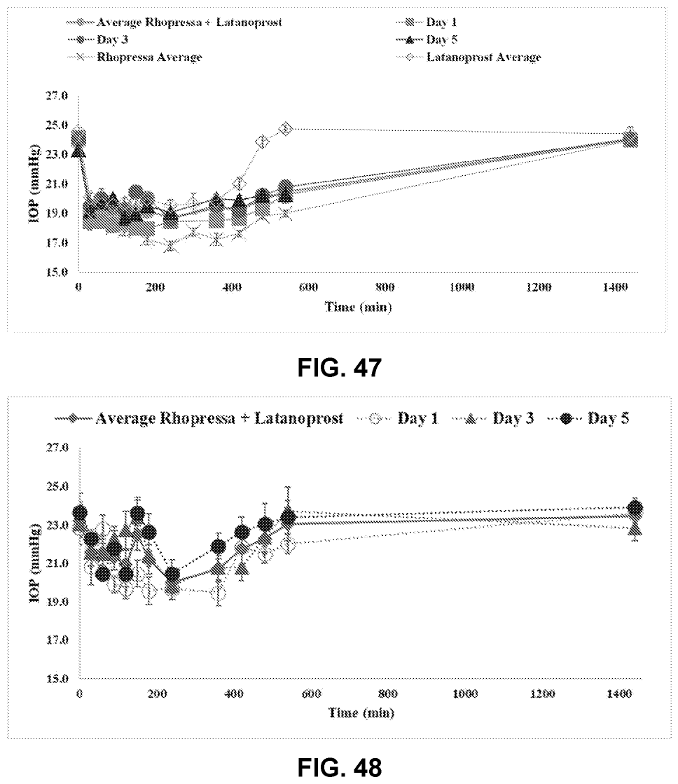

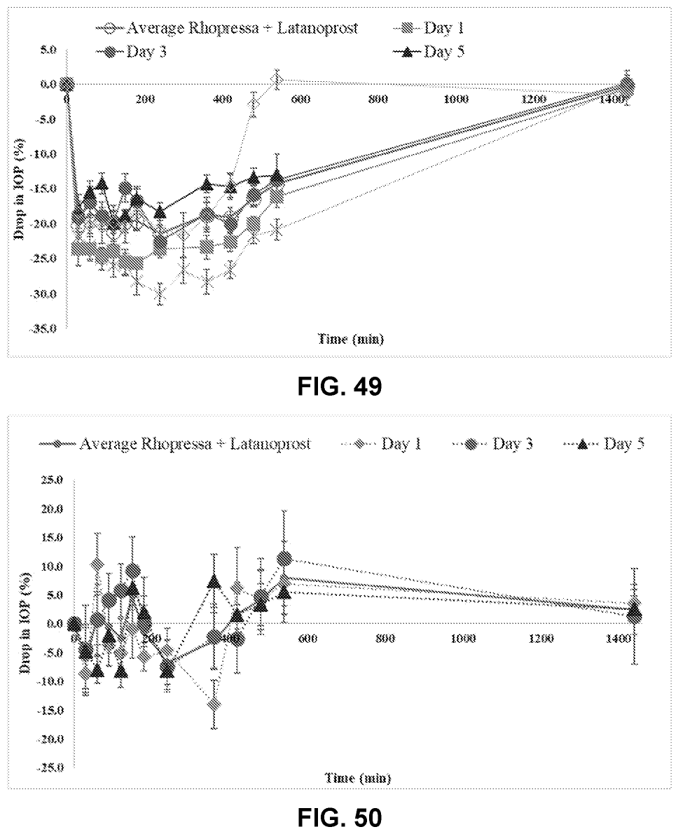

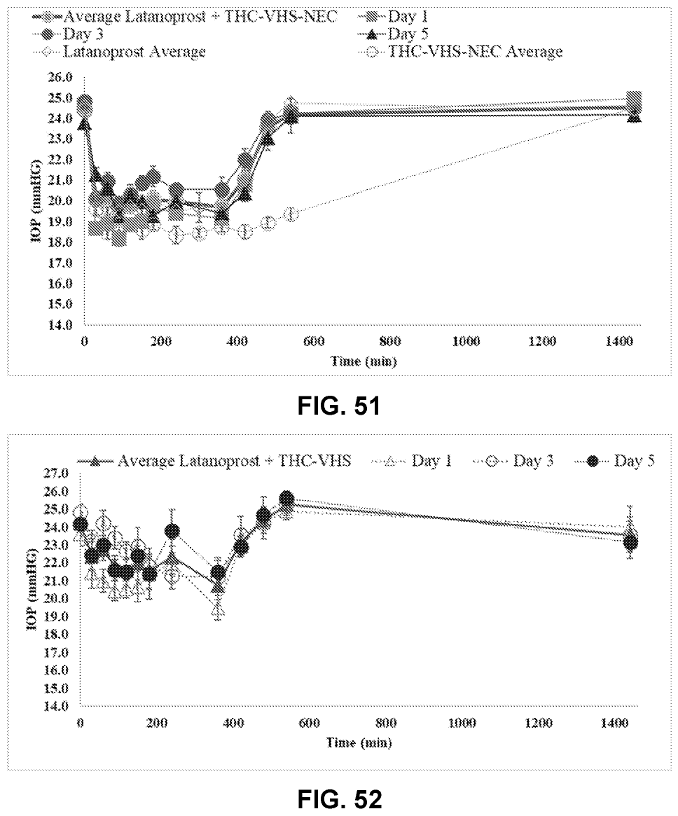

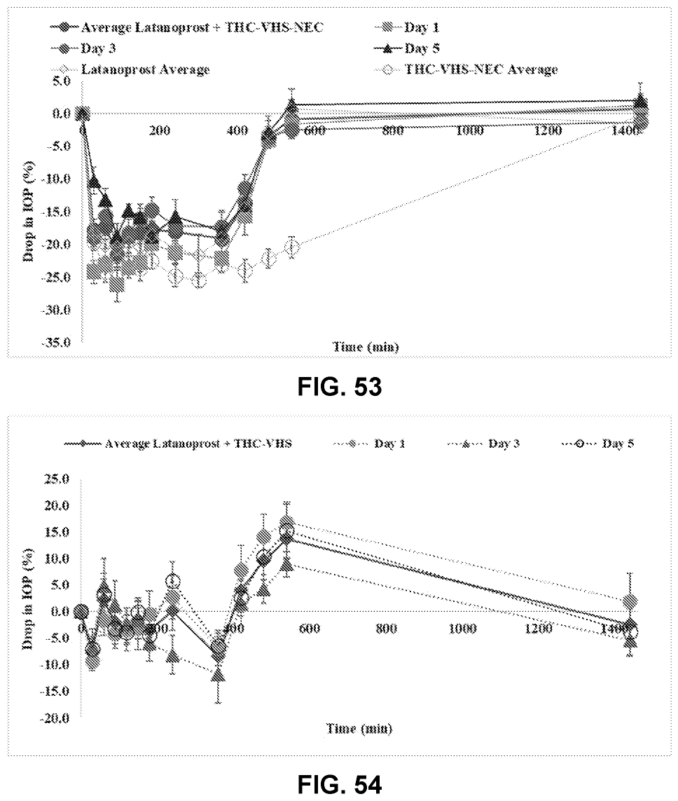

The data obtained from the co-administration studies are presented in FIGS. 27-54 (FIGS. 43-46: THC-VHS-NEC+Rhopressa®; FIGS. 47-50: Rhopressa®+Latanoprost; FIGS. 51-54: Latanoprost+THC-VHS-NEC). The comparative profiles from these studies are presented in FIGS. 27-52.

Administration was topical, with 50 μL of each treatment applied. The second drug was administered 15 minutes after the first drug in the left (treated eye). The right (contralateral) eye remained untreated. Results include the following:

THC-VHS-NEC formulation followed by Rhopressa® (FIGS. 43-46) demonstrated an average max drop in IOP of about 32.4+2.6% and maintained an average drop of 26.5+4.4% from 30 to 540 minutes. IOP returned to baseline within 24 h. The max drop in IOP occurred between 240 to 420 minutes but decreased in intensity over the 5 days from 8.6 to 7.0 mm Hg. However, at 30 minutes, the IOP had already dropped 20% below baseline on days 6 and 8, and by 90 minutes on day 10. Thus, the IOP lowering profile of the combination was better than that of THC-VHS-NEC formulation alone, or that of Rhopressa® alone. See also Table 10 below.

Rhopressa® followed by Latanoprost (FIGS. 47-50) demonstrated a max drop in IOP of about 22.7±2.9% and maintained an average drop of 19.0±2.1% from 30 to 540 minutes. The time for max drop in IOP showed some variability across the 3 days—180, 420, and 120 mins on days 6, 8 and 10, respectively. On day 6 the largest drop in IOP of 25.7±0.8% was observed, whereas on days 8 and 10 the drop in IOP was around 22.5±1.3 and 19.9±2.6%, respectively. The combination was thus similar to Latanoprost alone, but the activity was significantly inferior to that of Rhopressa® alone. The duration of action is at least 540 minutes, at which time the IOP still remained about 15% below baseline and returns to baseline by 24h. Thus, the duration of action of Latanoprost increased on combining with Rhopressa®, but Rhopressa® alone performed better than the combination with Latanoprost. See also Table 11 below.

Latanoprost followed by THC-VHS-NEC formulation (FIGS. 51-54) demonstrated a max drop in IOP of about 21.6±2.9% and maintained an average drop of 18.1±2.1% from 30 to 480 minutes. The max drop in IOP occurred around 90 minutes on all 3 days measured. However, on day 6 it was observed to have the larger max drop of 26.1±2.6% and day 8 and 10 the drop in IOP 19.9±1.3 and 18.7±2.0%, respectively. The IOP returns to baseline at 540 minutes. The IOP lowering profile of this combination was similar to that of Latanoprost alone. Thus, the activity of THC-VHS-NEC is not evident on combination with Latanoprost. See also Table 12 below.

TABLE 10
THC-VHS-NEC + Rhopressa ® IOP and % Drop in IOP
Average THC-VHS-NEC + Rhopressa ®

| Min | 0 | 30 | 60 | 90 | 120 | 150 | 180 | 240 | 360 | 42 | 480 | 540 | 1440 |
|---|---|---|---|---|---|---|---|---|---|---|---|---|---|
| Test eye | 23.7 | 19.3 | 19.0 | 17.9 | 17.9 | 17.2 | 16.7 | 16.3 | 16.3 | 16.3 | 17.1 | 17.5 | 22.9 |
| SEM | 0.3 | 0.3 | 0.2 | m | 0.3 | 0.5 | 0.5 | 0.5 | 0.3 | 0.4 | 0.2 | 0.2 | 0.3 |
| Drop in IOP | 0.0 | −18.4 | −19.9 | −24.2 | −24.4 | −27.5 | −29.4 | −31.3 | −31.2 | −31.2 | −27.9 | −26.2 | −3.4 |
| SEM | 0.0 | 1.7 | 1.4 | 2.0 | 1.6 | 2.2 | 2.0 | 2.2 | 1.5 | 1.8 | 1.3 | 1.2 | 1.8 |

Day 1

| Min | 0 | 30 | 60 | 90 | 120 | 150 | 180 | 240 | 360 | 420 | 480 | 540 | 1440 |
|---|---|---|---|---|---|---|---|---|---|---|---|---|---|
| Test Eye | 24.3 | 19.1 | 18.9 | 18.8 | 18.3 | 18.0 | 16.9 | 16.6 | 15.8 | 15.8 | 16.3 | 17.2 | 24.0 |
| SEM | 0.2 | 0.3 | 0.1 | 0.2 | 0.3 | 0.4 | 0.5 | 0.8 | 0.4 | 0.3 | 0.2 | 0.2 | 0.1 |
| Drop in IOP | 0.0 | −21.7 | −22.3 | −22.6 | −24.6 | −26.0 | −30.4 | −31.8 | −35.2 | −35.2 | −33.1 | −29.2 | −1.3 |
| SEM | 0.0 | 1.3 | 0.8 | 1.2 | 1.2 | 1.4 | 1.4 | 2.9 | 1.6 | 1.2 | 0.8 | 0.6 | 1.0 |

Day 3

| Min | 0 | 30 | 60 | 90 | 120 | 150 | 180 | 240 | 360 | 420 | 480 | 540 | 1440 |
|---|---|---|---|---|---|---|---|---|---|---|---|---|---|
| Test eye | 23.3 | 18.9 | 18.7 | 16.9 | 17.0 | 16.2 | 16.5 | 15.8 | 16.7 | 16.7 | 17.3 | 17.5 | 20.8 |
| SEM | 0.2 | 0.4 | 0.3 | 0.6 | 0.4 | 0.5 | 0.5 | 0.3 | 0.3 | 0.4 | 0.2 | 0.3 | 0.5 |
| Drop in IOP | 0.0 | −19.0 | −20.0 | −27.4 | −27.2 | −30.5 | −29.3 | −32.2 | −28.5 | −28.6 | −25.9 | −25.0 | −10.8 |
| SEM | 0.0 | 1.8 | 1.5 | 2.4 | 1.7 | 2.2 | 2.3 | 0.9 | 1.5 | 1.7 | 1.5 | 1.0 | 1.8 |

Day 5

| Min | 0 | 30 | 60 | 90 | 120 | 150 | 180 | 240 | 360 | 420 | 480 | 540 | 1440 |
|---|---|---|---|---|---|---|---|---|---|---|---|---|---|
| Test eye | 23.4 | 20.0 | 19.3 | 18.1 | 18.4 | 17.3 | 16.8 | 16.4 | 16.4 | 16.4 | 17.6 | 17.7 | 23.8 |
| SEM | 0.4 | 0.2 | 0.3 | 0.2 | 0.2 | 0.6 | 0.5 | 0.5 | 0.2 | 0.5 | 0.2 | 0.2 | 0.3 |
| Drop in IOP | 0.0 | −14.5 | −17.4 | −22.8 | −21.4 | −25.9 | −28.4 | −29.9 | −29.7 | −30.0 | −24.8 | −24.2 | 1.9 |
| SEM | 0.0 | 2.2 | 2.1 | 2.2 | 2.0 | 3.1 | 2.2 | 2.6 | 1.4 | 2.5 | 1.5 | 1.9 | 2.5 |

TABLE 11
Rhopressa ® + Latanoprost IOP and % Drop in IOP
Average Rhopressa ® + Latanoprost

| Min | 0 | 30 | 60 | 90 | 120 | 150 | 180 | 240 | 360 | 420 | 480 | 540 | 1440 |
|---|---|---|---|---|---|---|---|---|---|---|---|---|---|
| Test eye | 23.9 | 19.0 | 19.4 | 19.2 | 18.8 | 19.1 | 19.1 | 18.7 | 19.4 | 19.3 | 19.9 | 20.4 | 24.0 |
| SEM | 0.3 | 0.4 | 0.4 | 0.3 | 0.3 | 0.3 | 0.2 | 0.2 | 0.3 | 0.2 | 0.3 | 0.5 | 0.2 |
| Drop in IOP | 0.0 | −20.2 | −18.7 | −19.2 | −21.2 | −19.7 | −19.6 | −21.5 | −18.7 | −19.1 | −16.4 | −14.3 | −0.2 |
| SEM | 0.0 | 1.8 | 1.8 | 1.5 | 1.7 | 1.5 | 1.4 | 1.3 | 1.8 | 1.4 | 1.2 | 1.8 | 1.6 |

Day 1

| Min | 0 | 30 | 60 | 90 | 120 | 150 | 180 | 240 | 360 | 420 | 480 | 540 | 1440 |
|---|---|---|---|---|---|---|---|---|---|---|---|---|---|
| Test Eye | 24.2 | 18.4 | 18.4 | 18.2 | 18.4 | 18.0 | 17.9 | 18.4 | 18.5 | 18.7 | 19.7 | 20.3 | 24.0 |
| SEM | 0.2 | 0.6 | 0.4 | 0.4 | 0.3 | 0.4 | 0.2 | 0.2 | 0.3 | 0.2 | 0.5 | 0.4 | 0.2 |
| Drop in IOP | 0.0 | −23.7 | −23.6 | −24.6 | −23.9 | −25.5 | −25.7 | −23.6 | −23.4 | −22.7 | −18.4 | −16.1 | −0.6 |
| SEM | 0.0 | 2.4 | 1.7 | 1.2 | 1.0 | 1.7 | 0.8 | 1.3 | 1.7 | 1.3 | 2.0 | 1.6 | 1.3 |

Day 3

| Min | 0 | 30 | 60 | 90 | 120 | 150 | 180 | 240 | 360 | 420 | 480 | 540 | 1440 |
|---|---|---|---|---|---|---|---|---|---|---|---|---|---|
| Test eye | 24.1 | 19.4 | 20.0 | 19.5 | 19.3 | 20.4 | 20.0 | 18.6 | 19.6 | 19.2 | 20.2 | 20.8 | 24.1 |
| SEM | 0.4 | 0.4 | 0.7 | 0.4 | 0.2 | 0.4 | 0.2 | 0.2 | 0.3 | 0.2 | 0.2 | 0.5 | 0.2 |
| Drop in IOP | 0.0 | −19.2 | −16.9 | −18.9 | −19.7 | −14.9 | −16.7 | −22.5 | −18.5 | −20.0 | −15.8 | −13.7 | 0.2 |
| SEM | 0.0 | 1.0 | 2.2 | 1.9 | 1.6 | 2.0 | 2.0 | 1.3 | 2.3 | 1.2 | 1.4 | 0.9 | 1.8 |

Day 5

| Min | 0 | 30 | 60 | 90 | 120 | 150 | 180 | 240 | 360 | 420 | 480 | 540 | 1440 |
|---|---|---|---|---|---|---|---|---|---|---|---|---|---|
| Test eye | 23.3 | 19.2 | 19.7 | 20.0 | 18.7 | 18.9 | 19.5 | 19.1 | 20.0 | 19.9 | 20.2 | 20.3 | |
| SEM | 0.2 | 0.4 | 0.3 | 0.2 | 0.5 | 0.3 | 0.2 | 0.2 | 0.3 | 0.3 | 0.2 | 0.6 | |
| Drop in IOP | 0.0 | −17.8 | −15.4 | −14.2 | −19.9 | −18.8 | −16.4 | −18.3 | −14.3 | −14.7 | −13.3 | −13.0 | |
| SEM | 0.0 | 2.0 | 1.5 | 1.5 | 2.6 | 0.8 | 1.3 | 1.3 | 1.3 | 1.8 | 1.2 | 2.9 | |

TABLE 12
Latanoprost + THC-VHS-NEC IOP and % Drop in IOP
Average Latanoprost + THC-VHS-NEC

| Min | 0 | 30 | 60 | 90 | 120 | 150 | 180 | 240 | 360 | 420 | 480 | 540 | 1440 |
|---|---|---|---|---|---|---|---|---|---|---|---|---|---|
| Test eye | 24.4 | 20.0 | 20.1 | 19.1 | 19.8 | 19.9 | 20.1 | 20.0 | 19.7 | 21.1 | 23.6 | 24.2 | 24.5 |
| SEM | 0.4 | 0.3 | 0.3 | 0.4 | 0.4 | 0.3 | 0.3 | 0.2 | 0.4 | 0.5 | 0.4 | 0.5 | 0.4 |
| Drop in IOP | 0.0 | −17.8 | −17.3 | −21.6 | −18.9 | −18.3 | −17.8 | −18.1 | −19.2 | −13.7 | −3.5 | −0.9 | 0.6 |
| SEM | 0.0 | 1.7 | 1.9 | 2.0 | 1.6 | 1.6 | 1.8 | 1.7 | 2.5 | 2.4 | 1.5 | 1.9 | 2.1 |

Day 1

| Min | 0 | 30 | 60 | 90 | 120 | 150 | 180 | 240 | 360 | 420 | 480 | 540 | 1440 |
|---|---|---|---|---|---|---|---|---|---|---|---|---|---|
| Test Eye | 24.7 | 18.7 | 18.9 | 18.2 | 18.8 | 19.0 | 19.7 | 19.4 | 19.2 | 21.1 | 23.7 | 24.2 | 24.9 |
| SEM | 0.4 | 0.3 | 0.3 | 0.4 | 0.2 | 0.2 | 0.2 | 0.2 | 0.3 | 0.8 | 0.3 | 0.2 | 0.3 |
| Drop in IOP | 0.0 | −24.2 | −23.2 | −26.1 | −23.5 | −22.8 | −19.9 | −21.2 | −22.1 | −14.5 | −4.0 | −1.6 | 1.3 |
| SEM | 0.0 | 1.7 | 2.5 | 2.6 | 1.7 | 1.3 | 1.7 | 1.7 | 2.0 | 3.3 | 0.7 | 1.9 | 1.8 |

Day 3

| Min | 0 | 30 | 60 | 90 | 120 | 150 | 180 | 240 | 360 | 420 | 480 | 540 | 1440 |
|---|---|---|---|---|---|---|---|---|---|---|---|---|---|
| Test eye | 24.8 | 20.1 | 20.9 | 19.9 | 20.3 | 20.8 | 21.2 | 20.6 | 20.6 | 22.2 | 23.9 | 24.2 | 24.5 |
| SEM | 0.1 | 0.3 | 0.4 | 0.3 | 0.5 | 0.4 | 0.5 | 0.2 | 0.6 | 0.5 | 0.4 | 0.4 | 0.4 |
| Drop in IOP | 0.0 | −19.0 | −15.7 | −19.9 | −18.3 | −16.1 | −14.8 | −17.2 | −17.3 | −10.7 | −3.6 | −2.5 | −1.3 |
| SEM | 0.0 | 1.3 | 1.7 | 1.3 | 2.2 | 1.6 | 2.0 | 0.7 | 2.1 | 2.3 | 1.3 | 1.4 | 1.7 |

Day 5

| Min | 0.0 | 30 | 60 | 90 | 120 | 150 | 180 | 240 | 360 | 420 | 480 | 540 | 1440 |
|---|---|---|---|---|---|---|---|---|---|---|---|---|---|
| Test eye | 23.8 | 21.3 | 20.6 | 19.3 | 20.2 | 19.9 | 19.3 | 19.9 | 19.4 | 20.4 | 23.1 | 24.1 | 24.2 |
| SEM | 0.6 | 0.3 | 0.3 | 0.4 | 0.4 | 0.3 | 0.3 | 0.3 | 0.4 | 0.4 | 0.6 | 0.8 | 0.4 |
| Drop in IOP | 0.0 | −10.3 | −13.1 | −18.7 | −14.9 | −15.9 | −18.7 | −15.8 | −18.1 | −14.0 | −2.9 | 1.4 | 2.0 |
| SEM | 0.0 | 2.0 | 1.6 | 2.0 | 0.9 | 2.0 | 1.8 | 2.7 | 3.2 | 2.4 | 2.4 | 2.4 | 2.7 |

Thus, Latanoprost decreased the efficacy of both THC-VHS-NEC and Rhopressa® when combined. This could be because of changes in elimination profiles and/or changes in metabolism. Overall, the combination of THC-VHS-NEC formulation and Rhopressa® was the most effective in terms of both duration of activity as well as max drop in IOP, compared to all other single and combination treatment studied. Results are summarized in Table 13 below:

TABLE 13
Max Drop in IOP and Max % Drop in
IOP for Multiple Drug Formulations

| | | Day 6 | | Day 8 | Day 10 |
|---|---|---|---|---|---|
| THC-VHS-NEC + | Max IOP drop | 8.6 | | 7.5 | 7 |
| Rhopressa | (mmHg) | 360-420 | min | 240 min | 240 min |
| | Max IOP drop | −35.2 | | −32.2 | −29.9 |
| | (%) | 360-420 | min | 240 min | 240 min |
| Rhopressa + | Max IOP drop | 6.2 | | 4.7 | 4.7 |
| Latanoprost | (mmHg) | 180 | min | 420 min | 120 min |
| | Max IOP drop | −25.7 | | −19.6 | −19.9 |
| | (%) | 180 | min | 420 min | 120 min |
| Latanoprost + | Max IOP drop | 6.5 | | 4.9 | 4.5 |
| THC-VHS-NEC | (mmHg) | 90 | | 90 | 90 min |
| | Max IOP drop | −26.1 | | −19.9 | −18.7 |
| | (%) | 90 | min | 90 min | 90 min |

Overall, the IOP lowering profile of THC-VHS-NEC in the Dutch-Belted rabbits was similar to the commercially available Rhopressa® in terms of duration of action, and superior to latanoprost in terms of both intensity and duration of action. THC-VHS-NEC formulation followed by Rhopressa® was most effective, in terms of the IOP lowering profile, amongst all the formulations and combinations tested.

What is claimed:

1. A method for treating or preventing elevated intraocular pressure (IOP) in a subject in need thereof, the method comprising administering to the subject a delta-9-tetrahydrocannabinol amino acid ester or derivative thereof and a Rho kinase inhibitor.

2. The method of claim 1, wherein the Rho kinase inhibitor is netarsudil or a pharmaceutically acceptable salt thereof.

3. The method of claim 1, wherein the delta-9-tetrahydrocannabinol amino acid ester has the structure I: 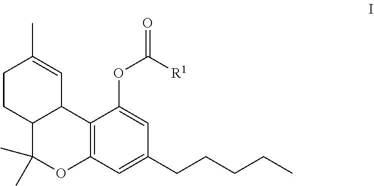
wherein R¹ comprises valine, sarcosine, leucine, glutamine, tryptophan, tyrosine, alanine, 4(4-aminophenyl)butyric acid, or a salt thereof.

4. The method of claim 1, wherein the derivative of the delta-9-tetrahydrocannabinol amino acid ester has the structure II 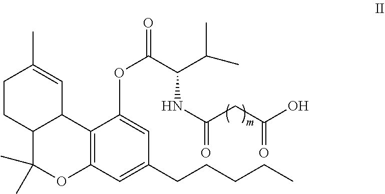
where n is an integer from 1 to 9.

5. The method of claim 1, wherein the derivative of the delta-9-tetrahydrocannabinol amino acid ester is delta-9-tetrahydrocannabinol-valine-hemisuccinate.

6-8. (canceled)

9. A nanoemulsion comprising a delta-9-tetrahydrocannabinol amino acid ester or derivative thereof, an ophthalmically suitable oil, an ophthalmically suitable surfactant, and water.

10. The nanoemulsion of claim 9, wherein the delta-9-tetrahydrocannabinol amino acid ester has the structure I: 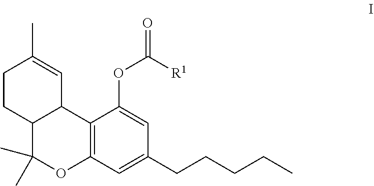
wherein R¹ comprises valine, sarcosine, leucine, glutamine, tryptophan, tyrosine, alanine, 4(4-aminophenyl)butyric acid, or a salt thereof.

11. The nanoemulsion of claim 9, wherein the derivative of the delta-9-tetrahydrocannabinol amino acid ester has the structure II 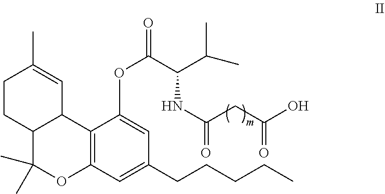
where n is an integer from 1 to 9.

12. (canceled)

13. The nanoemulsion of claim 9, wherein the nanoemulsion comprises from about 0.01% w/v to about 2% w/v of the delta-9-tetrahydrocannabinol amino acid ester or derivative thereof.

14. The nanoemulsion of claim 9, wherein the ophthalmically suitable oil comprises castor oil, cottonseed oil, soybean oil, sesame oil, or any combination thereof.

15. The nanoemulsion of claim 9, wherein the nanoemulsion comprises from about 1% to about 10% w/v of the ophthalmically suitable oil.

16. The nanoemulsion of claim 9, wherein the ophthalmically suitable surfactant comprises a nonionic surfactant, wherein the ophthalmically suitable surfactant comprises a poloxamer, a polysorbate, or any combination thereof.

17. (canceled)

18. The nanoemulsion of claim 16, wherein the nanoemulsion comprises from about 0.01% w/v to about 1% w/v of the poloxamer and from about 0.5% w/v to about 5% w/v of the polysorbate.

19. The nanoemulsion of claim 9, further comprising an ophthalmically suitable polymer, wherein the ophthalmically suitable polymer comprises a crosslinked polyacrylic acid.

20. (canceled)

21. The nanoemulsion of claim 9, further comprising an ophthalmically suitable polyol, wherein the ophthalmically suitable polyol comprises glycerin.

22. (canceled)

23. The nanoemulsion of claim 9, further comprising an ophthalmically suitable ethoxylated tocopherol or tocotrienol.

24. (canceled)

25. The nanoemulsion of claim 23, wherein the nanoemulsion comprises from about 0.0001% w/v to about 0.01% w/v of the ophthalmically suitable ethoxylated tocopherol or tocotrienol.

26. A nanoemulsion comprising from about 0.01% w/v to about 2% w/v of a delta-9-tetrahydrocannabinol amino acid ester or derivative thereof, from about 1% to about 10% w/v of an ophthalmically suitable oil, from about 0.51% w/v to about 6% w/v of an ophthalmically suitable surfactant, from about 0.1% w/v to about 2% w/v of an ophthalmically suitable polymer, from about 1% to about 5% w/v of an ophthalmically suitable polyol, from about 0.0001% w/v to about 0.01% w/v of an ophthalmically suitable ethoxylated tocopherol or tocotrienol, and water.

27. The nanoemulsion of claim 26, further comprising a Rho kinase inhibitor, wherein the Rho kinase inhibitor is netarsudil or a pharmaceutically acceptable salt thereof.

28. (canceled)

29. The nanoemulsion of claim 27, wherein the nanoemulsion comprises from about 0.005% w/v to about 0.05% w/v of the Rho kinase inhibitor.

30-33. (canceled)
